(19) United States
(12) Patent Application Publication
(10) Pub. No.: US 2003/0104472 A1
(43) Pub. Date: Jun. 5, 2003

(54) Antibody specifically binding human pinch protein homolog

(76) Inventors: Preeti G. Lal, Santa Clara, CA (US); Karl J. Guegler, Menlo Park, CA (US); Neil C. Corley, Castro Valley, CA (US)

Correspondence Address: INCYTE GENOMICS, INC., 3160 PORTER DRIVE, PALO ALTO, CA, 94304, US

(21) Appl. No.: 10/092,066

(22) Filed: Mar. 4, 2002

Related U.S. Application Data
(63) Continuation-in-part of application No. 09/528,959, filed on Mar. 20, 2000, now Pat. No. 6,379,904.
Application No. 09/528,959 is a division of application No. 09/008,465, filed on Jan. 16, 1998, now Pat. No. 6,174,702.

(51) Int. Cl. G01N 33/53; C12P 21/04; C12N 9/00; C07K 16/40

(52) U.S. Cl. 435/7.1; 435/70.21; 435/183; 530/388.26

(57) ABSTRACT

The invention provides a human PINCH protein homolog (PINCH-PH), polynucleotides encoding the protein and antibodies which specifically bind the protein. The invention also provides expression vectors, host cells, agonists, antagonists and compositions for diagnosing or treating disorders associated with expression of the protein.

This application is a continuation-in-part of U.S. Ser. No. 09/528,959, filed Mar. 20, 2000, which was a divisional of U.S. Pat. No 6,174,702, issued Jan. 16, 2001, which matured from U.S. Ser. No. 09/008,465, filed Jan. 16, 1998.

### FIELD OF THE INVENTION

This invention relates to a human PINCH protein homolog, polynucleotides encoding the protein, and an antibody that specifically binds the protein which may be used to diagnose, to stage, to treat, or to monitor the progression or treatment of cancer and reproductive disorders.

### BACKGROUND OF THE INVENTION

LIM proteins are a family of proteins that share a common structural domain. The LIM motif is so named because it was first described in three proteins from Drosophila melanogaster designated L, I, and M. The LIM motif is a cysteine-rich region with a characteristic pattern: [C—X—X—C—X_{17±1}—H—X—X—C]—X—X—[C—X—X—C—X_{17±1}—C—X—X—C]. LIM motifs form two loop structures and coordinate a zinc ion within each loop.

The LIM motif has been identified in a variety of proteins including transcription factors, cytoskeletal proteins, and signaling molecules. LIM proteins are involved in cell fate determination, growth regulation, and oncogenesis. At least twenty-three members of the LIM family have been described in species as diverse as nematodes and humans. Some LIM proteins consist of one, two, or three repeats of the LIM motif (LIM-only proteins). Others contain a LIM motif with a homeodomain (LIM-HD proteins) or a protein kinase domain (LIM-PK). LIM-PK inhibits the Ras oncogene-mediated differentiation of neural PC12 cells. LIM-HD proteins interact with DNA as well as bind to other proteins and are implicated in the control of differentiation of specific cell types. Studies in C. elegans demonstrated that LIM-HD proteins are involved in control of cell differentiation. Lin-11, a LIM-HD protein, controls the asymmetric cell divisions during vulval development, while Mec-3 is required for the differentiation of mechanosensory neurons (Way and Chalfie (1988) Cell 54:5-16; Freyd et al. (1990) Nature 344:876-879).

The LIM-only proteins have not been shown to bind DNA, although LIM structure is similar to the zinc finger, a well-characterized DNA-binding domain. LIM-only proteins include the rat cysteine-rich intestinal protein (CRIP), the human RBTN1 and RBTN2 proteins, and the chicken zyxin protein (Higuchi et al. (1997) Oncogene 14:1819-1825; Sanchez-Garcia and Rabbitts (1994) Trends Genet 10:315-320; and Dawid et al. (1995) CR Acad Sci III 318:295-306). The genes for RBTN1 and RBTN2 are located on chromosome 11. Translocation mutations of chromosome 11 are associated with specific human T-cell acute leukemias. Transgenic expression of RBTN1 or RBTN2 produces leukemia and lymphoma in mice (McGuire et al. (1992) Mol Cell Biol 12:4186-4196; Fisch et al. (1992) Oncogene 7:2389-2397).

A LIM-only protein known as PINCH protein (particularly interesting new Cys-His protein) was recently cloned from a human fetal liver library. PINCH protein contains five repeats of the LIM motif. Messenger RNA for PINCH protein is widely expressed, particularly in reproductive tissues, heart, and peripheral blood leukocytes (Rearden (1994) Biochem Biophys Res Commun 201:1124-1131).

The discovery of a new human PINCH protein homolog, polynucleotides encoding the protein and an antibody that specifically binds the protein satisfies a need in the art by providing new compositions which may be used to diagnose, to stage, to treat, or to monitor the progression or treatment of cancer and reproductive disorders.

### SUMMARY OF THE INVENTION

The invention provides a purified human PINCH protein homolog (PINCH-PH), comprising a protein having the amino acid sequence of SEQ ID NO:1 or a fragment of SEQ ID NO:1. The invention also provides a purified variant of PINCH-PH having at least 90% sequence identity to the sequence of SEQ ID NO:1. The invention further provides isolated polynucleotides, which encode the protein comprising the amino acid sequence of SEQ ID NO:1, or the complements thereof. The invention still further provides an isolated polynucleotide having at least 90% sequence identity to the polynucleotide encoding the protein having the amino acid sequence of SEQ ID NO:1. The invention yet still further provides an isolated and purified polynucleotide, which hybridizes under stringent conditions to the polynucleotide encoding the protein comprising the sequence of SEQ ID NO:1, or the complements thereof.

The invention provides an isolated polynucleotide comprising a nucleic acid sequence of SEQ ID NO:2 or a fragment of SEQ ID NO:2, and an isolated and purified polynucleotide variant having at least 90% sequence identity to the polynucleotide comprising the nucleic acid sequence of SEQ ID NO:2 or the complements thereof. The invention also provides an expression vector containing at least a fragment of the polynucleotide encoding the protein comprising the amino acid sequence of SEQ ID NO:1 or a fragment of SEQ ID NO:1. In another aspect, the expression vector is contained within a host cell. The invention further provides a method for producing a protein, the method comprising culturing the host cell containing an expression vector containing at least a fragment of a polynucleotide encoding PINCH-PH under conditions for the expression of the protein; and recovering the protein from the host cell culture.

The invention provides a purified antibody which specifically bind the protein having the amino acid sequence of SEQ ID NO:1. The invention also provides a method for using a protein or an immunogenic fragment thereof to screen a plurality of antibodies to identify an antibody which specifically binds the protein comprising contacting a plurality of antibodies with the protein under conditions to form an antibody:protein complex, and dissociating the antibody from the antibody:protein complex, thereby obtaining antibody which specifically binds the protein. The invention further provides methods for using a protein to prepare and purify polyclonal and monoclonal antibodies which specifically bind the protein. The method for preparing a polyclonal antibody comprises immunizing a animal with protein under conditions to elicit an antibody response, isolating animal antibodies, attaching the protein to a substrate, contacting the substrate with isolated antibodies under conditions to allow specific binding to the protein, dissociating the antibodies from the protein, thereby obtaining purified polyclonal antibodies. The method for preparing a monoclonal antibodies comprises immunizing a animal with a protein under conditions to elicit an antibody response, isolating antibody producing cells from the animal, fusing the antibody producing cells with immortalized cells in culture to form monoclonal antibody producing hybridoma cells, culturing the hybridoma cells, and isolating monoclonal antibodies from culture.

The invention provides a method for using an antibody to detect expression of a protein in a sample, the method comprising combining the antibody with a sample under conditions for formation of antibody:protein complexes; and detecting complex formation, wherein complex formation indicates expression of the protein in the sample. In one aspect, the sample is biopsied tissue. In another aspect, the amount of complex formation when compared to standards is diagnostic of a cancer or reproductive disorder, particularly prostatic adenocarcinoma and Hodgkin's disease. The invention also provides a method for immunopurification of a protein comprising attaching an antibody to a substrate, exposing the antibody to a sample containing protein under conditions to allow antibody:protein complexes to form, dissociating the protein from the complex, and collecting purified protein. The invention further provides an array containing an antibody which specifically binds the protein.

The invention provides compositions comprising an isolated polynucleotide which encodes the protein, a purified protein, or an isolated antibody, agonist or antagonist which specifically binds the protein and either a labeling moiety or a pharmaceutical agent.

### BRIEF DESCRIPTION OF THE FIGURES

FIGS. 1A, 1B, 1C, 1D, and 1E show the amino acid sequence (SEQ ID NO:1) and nucleic acid sequence (SEQ ID NO:2) of PINCH-PH. The alignment was produced using MACDNASIS PRO software (Hitachi Software Engineering, South San Francisco Calif.).

FIGS. 2A and 2B show the amino acid sequence alignments between PINCH-PH (3540806; SEQ ID NO:1) and PINCH protein (GI 516012; SEQ ID NO:3), produced using the MEGALIGN program of LASERGENE software (DNASTAR, Madison Wis.).

### DESCRIPTION OF THE INVENTION

It is understood that this invention is not limited to the particular machines, materials and methods described. It is also to be understood that the terminology used herein is for the purpose of describing particular embodiments and is not intended to limit the scope of the present invention which will be limited only by the appended claims. As used herein, the singular forms “a”, “an”, and “the” include plural reference unless the context clearly dictates otherwise. For example, a reference to “a host cell” includes a plurality of such host cells known to those skilled in the art.

Unless defined otherwise, all technical and scientific terms used herein have the same meanings as commonly understood by one of ordinary skill in the art to which this invention belongs. All publications mentioned herein are cited for the purpose of describing and disclosing the cell lines, protocols, reagents and vectors which are reported in the publications and which might be used in connection with the invention. Nothing herein is to be construed as an admission that the invention is not entitled to antedate such disclosure by virtue of prior invention.

### Definitions

“PINCH-PH” refers to the protein having the amino acid sequence of purified PINCH-PH obtained from any species, particularly a mammalian species, including bovine, ovine, porcine, murine, equine, and preferably the human species, from any source, whether natural, synthetic, semi-synthetic, or recombinant.

“Agonist” refers to a molecule which, when bound to PINCH-PH, increases or prolongs the biological or immunological activity of the protein. Agonists may include proteins, nucleic acids, carbohydrates, or any other molecules which bind to and modulate the protein.

“Antagonist” refers to a molecule which, when bound to a protein, decreases or shortens the biological or immunological activity of the protein. Antagonists may include proteins, nucleic acids, carbohydrates, antibodies, or any other molecules which bind to and modulate the protein.

“Antibody” refers to intact immunoglobulin molecule, a polyclonal antibody, a monoclonal antibody, a chimeric antibody, a recombinant antibody, a humanized antibody, single chain antibodies, a Fab fragment, an F(ab′)₂ fragment, an Fv fragment; and an antibody-peptide fusion protein.

“Antigenic determinant” refers to an antigenic or immunogenic epitope, structural feature, or region of an oligopeptide, peptide, or protein which is capable of inducing formation of an antibody which specifically binds the protein. Biological activity is not a prerequisite for immunogenicity.

“Array” refers to an ordered arrangement of at least two polynucleotides, proteins, or antibodies on a substrate. At least one of the polynucleotides, proteins, or antibodies represents a control or standard, and the other polynucleotide, protein, or antibody of diagnostic or therapeutic interest. The arrangement of at least two and up to about 40,000 polynucleotides, proteins, or antibodies on the substrate assures that the size and signal intensity of each labeled complex, formed between each polynucleotide and at least one nucleic acid, each protein and at least one ligand or antibody, or each antibody and at least one protein to which the antibody specifically binds, is individually distinguishable.

A “composition” refers to a polynucleotide, a protein or an antibody which specifically binds the protein and a pharmaceutical agent or carrier or a heterologous, labeling or purification moiety.

“Derivative” refers to a polynucleotide or a protein that has been subjected to a chemical modification. Derivatization of a polynucleotide can involve substitution of a nontraditional base such as queosine or of an analog such as hypoxanthine. These substitutions are well known in the art. Derivatization of a protein involves the replacement of a hydrogen by an acetyl, acyl, alkyl, amino, formyl, or morpholino group. Derivative molecules retain the biological activities of the naturally occurring molecules but may confer advantages such as longer lifespan or enhanced activity.

“Differential expression” refers to an increased, upregulated or present, or decreased, downregulated or absent, gene expression as detected by the absence, presence, or at least two-fold change in the amount of transcribed messenger RNA or translated protein in a sample.

An “expression profile” is a representation of gene expression in a sample. A nucleic acid expression profile is produced using sequencing, hybridization, or amplification technologies and mRNAs or polynucleotides from a sample. A protein expression profile, although time delayed, mirrors the nucleic acid expression profile and uses PAGE, ELISA, FACS, or arrays and labeling moieties or antibodies to detect expression in a sample. The nucleic acids, proteins, or antibodies may be used in solution or attached to a substrate, and their detection is based on methods and labeling moieties well known in the art.

“Identity”, as applied to sequences, refers to the quantification (usually percentage) of nucleotide or residue matches between at least two sequences aligned using a standardized algorithm such as Smith-Waterman alignment (Smith and Waterman (1981) J Mol Biol 147:195-197), CLUSTALW (Thompson et al. (1994) Nucleic Acids Res 22:4673-4680), or BLAST2 (Altschul et al. (1997) Nucleic Acids Res 25:3389-3402. BLAST2 may be used in a standardized and reproducible way to insert gaps in one of the sequences in order to optimize alignment and to achieve a more meaningful comparison between them. “Similarity” as applied to proteins uses the same algorithms but takes into account conservative substitutions of nucleotides or residues.

“Isolated” or “purified” refers to any molecule or compound that is separated from its natural environment and is from about 60% free to about 90% free from other components with which it is naturally associated.

“Labeling moiety” refers to any reporter molecule whether a visible or radioactive label, stain or dye that can be attached to or incorporated into a polynucleotide or protein. Visible labels and dyes include but are not limited to anthocyanins, β glucuronidase, BIODIPY, Coomassie blue, Cy3 and Cy5, digoxigenin, FITC, green fluorescent protein, luciferase, spyro red, silver, and the like. Radioactive markers include radioactive forms of hydrogen, iodine, phosphorous, sulfur, and the like.

“Modulate” refers to any change in the presence or activity of PINCH-PH in a cell or tissue. For example, modulation may cause an increase or a decrease in protein activity, binding characteristics, or any other biological, functional, or immunological properties of the protein.

“Polynucleotide” refers to an isolated cDNA, nucleic acid, or a fragment thereof, that contains from about 400 to about 12,000 nucleotides. It may have originated recombinantly or synthetically, may be double-stranded or single-stranded, represents coding and noncoding 3′ or 5′ sequence, generally lacks introns and may be purified or combined with carbohydrate, lipids, protein or inorganic elements or substances.

The phrase “polynucleotide encoding a protein” refers to a nucleic acid whose sequence closely aligns with sequences that encode conserved regions, motifs or domains identified by employing analyses well known in the art. These analyses include BLAST (Altschul (1993) J Mol Evol 36:290-300; Altschul et al. (1990) J Mol Biol 215:403-410) and BLAST2 (Altschul (1997) supra) which provide identity within the conserved region. Brenner et al. (1998, Proc Natl Acad Sci 95:6073-6078) who analyzed BLAST for its ability to identify structural homologs by sequence identity found 30% identity is a reliable threshold for sequence alignments of at least 150 residues and 40% is a reasonable threshold for alignments of at least 70 residues (Brenner, page 6076, column 2).

“Protein” refers to a polypeptide or any portion thereof. An “oligopeptide” is an amino acid sequence from about five residues to about 15 residues that is used as part of a fusion protein to produce an antibody.

“Sample” is used in its broadest sense as containing nucleic acids, proteins, antibodies, and the like. A sample may comprise a bodily fluid; the soluble fraction of a cell preparation, or an aliquot of media in which cells were grown; a chromosome, an organelle, or membrane isolated or extracted from a cell; genomic DNA, RNA, or cDNA in solution or bound to a substrate; a cell; a tissue or tissue biopsy; a tissue print; buccal cells, skin, a hair or its follicle; and the like.

“Specific binding” refers to a special and precise interaction between two molecules which is dependent upon their structure, particularly their molecular side groups. Examples include the intercalation of a regulatory protein into the major groove of a DNA molecule, the hydrogen bonding along the backbone between two single stranded nucleic acids, or the binding between an epitope of a protein and an agonist, antagonist, or antibody.

“Substrate” refers to any rigid or semi-rigid support to which polynucleotides, proteins or antibodies are bound and includes membranes, filters, chips, slides, wafers, fibers, magnetic or nonmagnetic beads, gels, capillaries or other tubing, plates, polymers, and microparticles with a variety of surface forms including wells, trenches, pins, channels and pores.

A “transcript image” (TI) is a profile of gene transcription activity in a particular tissue at a particular time. TI provides assessment of the relative abundance of expressed polynucleotides in the cDNA libraries of an EST database as described in U.S. Pat. No. 5,840,484, incorporated herein by reference.

“Variant” refers to molecules that are recognized variations of a polynucleotide or a protein encoded by the polynucleotide. Splice variants may be determined by BLAST score, wherein the score is at least 100, and most preferably at least 400. Allelic variants have high percent identity to the polynucleotides of the invention and may differ by about three bases per hundred bases. “Single nucleotide polymorphism” (SNP) refers to a change in a single base as a result of a substitution, insertion or deletion. The change may be conservative (purine for purine) or non-conservative (purine to pyrimidine) and may or may not result in a change in an encoded amino acid.

### THE INVENTION

The invention is based on the discovery of a new human PINCH protein homolog (PINCH-PH), the polynucleotides encoding PINCH-PH, and antibodies which specifically bind the protein which may be used to diagnose, to stage, to treat, or to monitor the progression or treatment of cancer and reproductive disorders, particularly adenocarcinoma of the prostate and Hodgkin's disease.

Nucleic acids encoding PINCH-PH of the present invention were first identified in Incyte Clone 3540806 from the seminal vesicle cDNA library (SEMVNOT04) using a computer search for amino acid sequence alignments. A consensus sequence, SEQ ID NO:2, was derived from the following overlapping and/or extended nucleic acid sequences: Incyte Clones 3540806 (SEMVNOT04), 853536 (NGANNOT01), 2190641 (THYRTUT03), 776025 (COLNNOT05), 1703222 (DUODNOT02), 1722945 (BLADNOT060, and 1262471 (SYNORAT05).

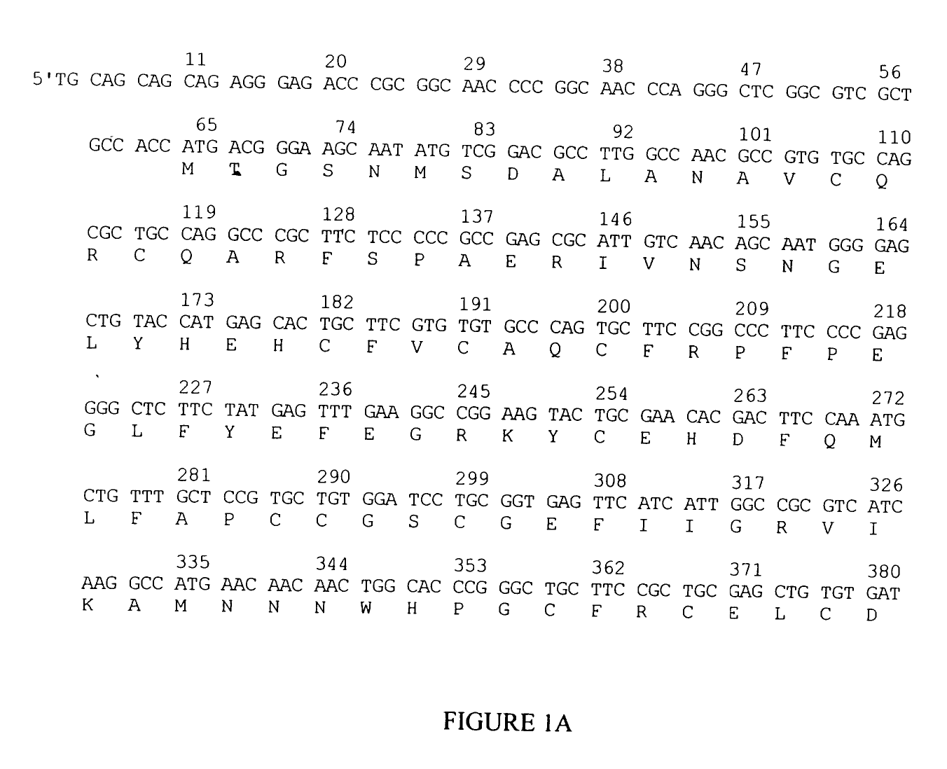

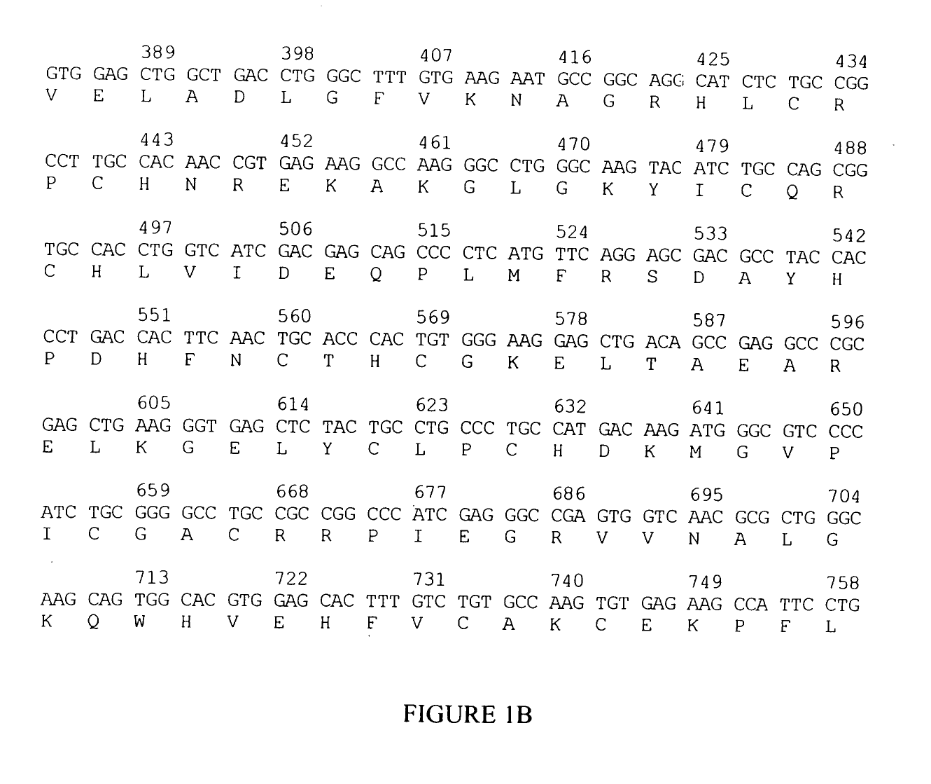

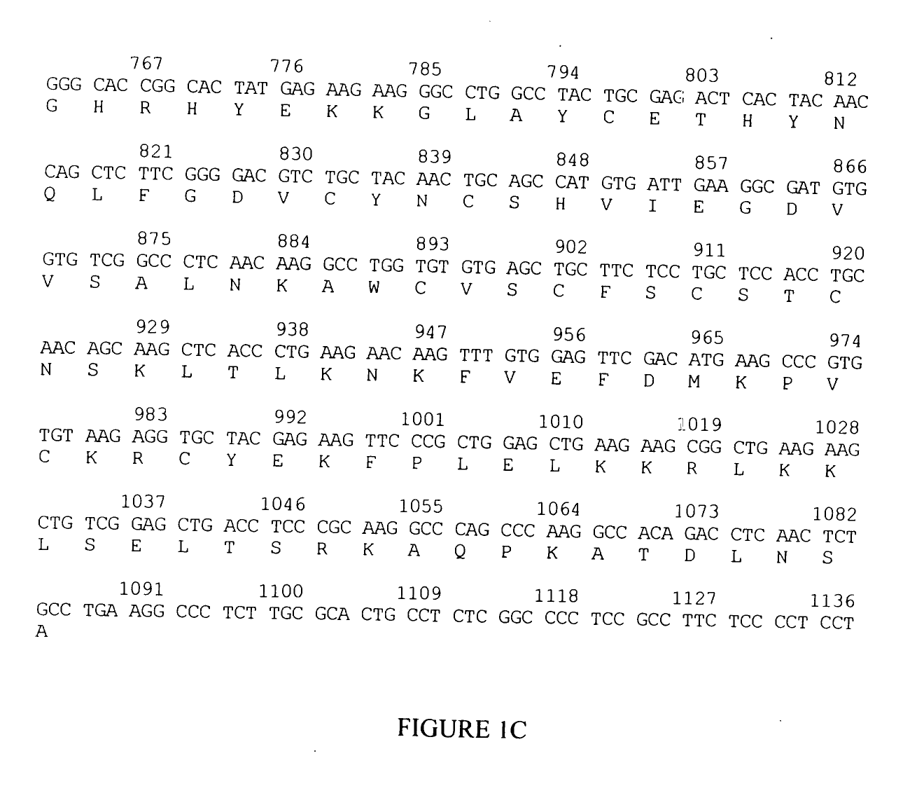

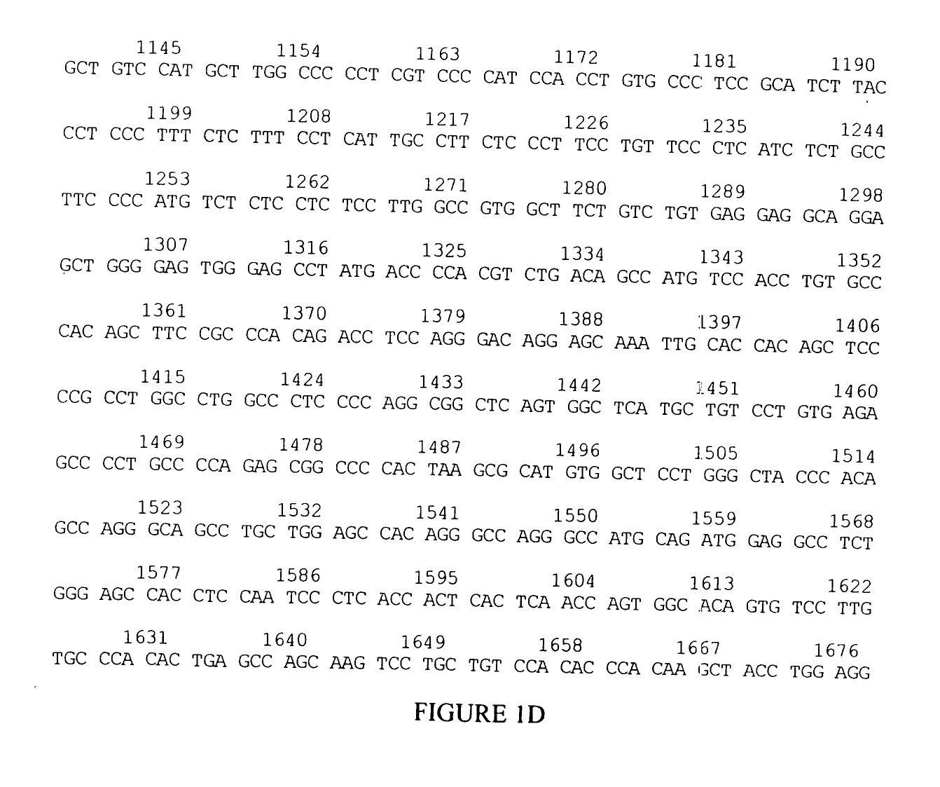

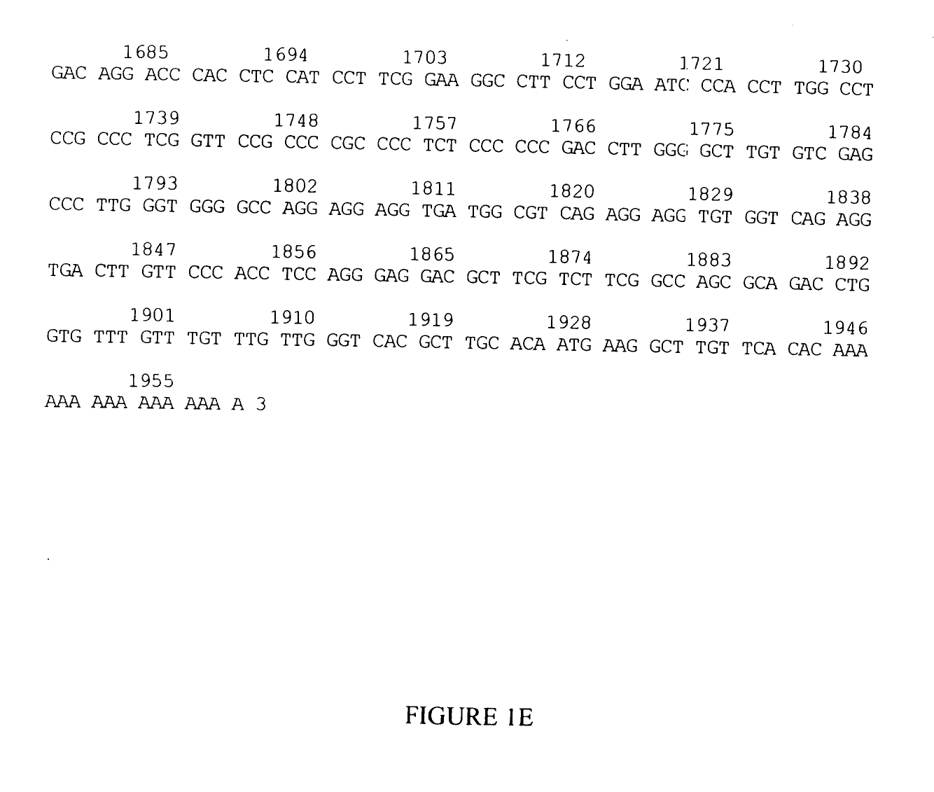

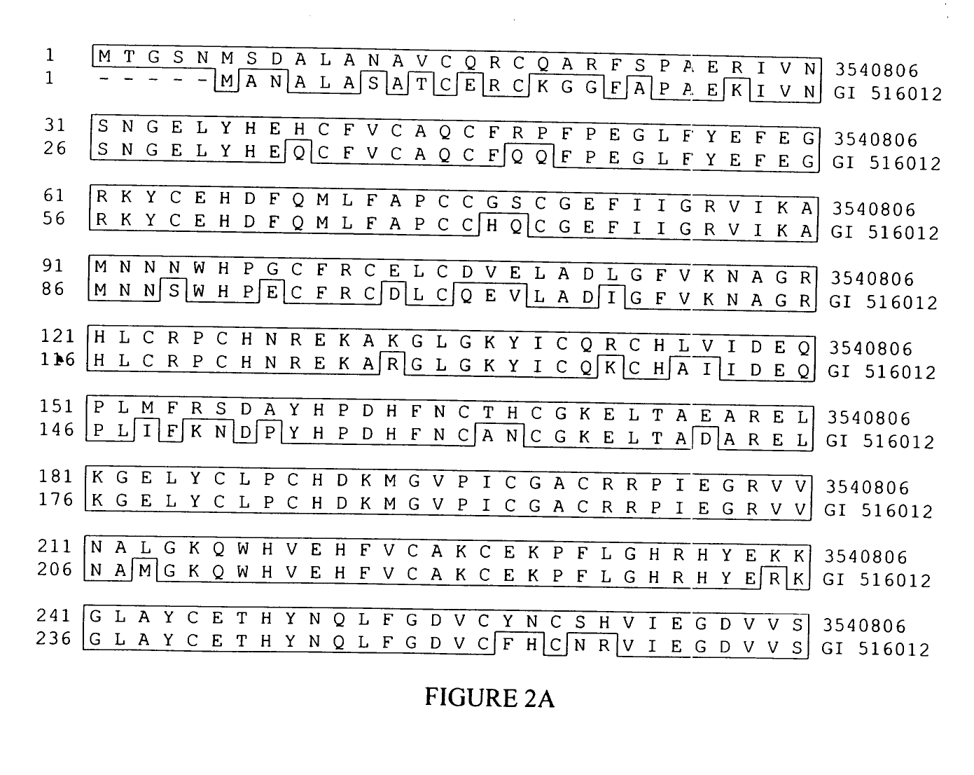

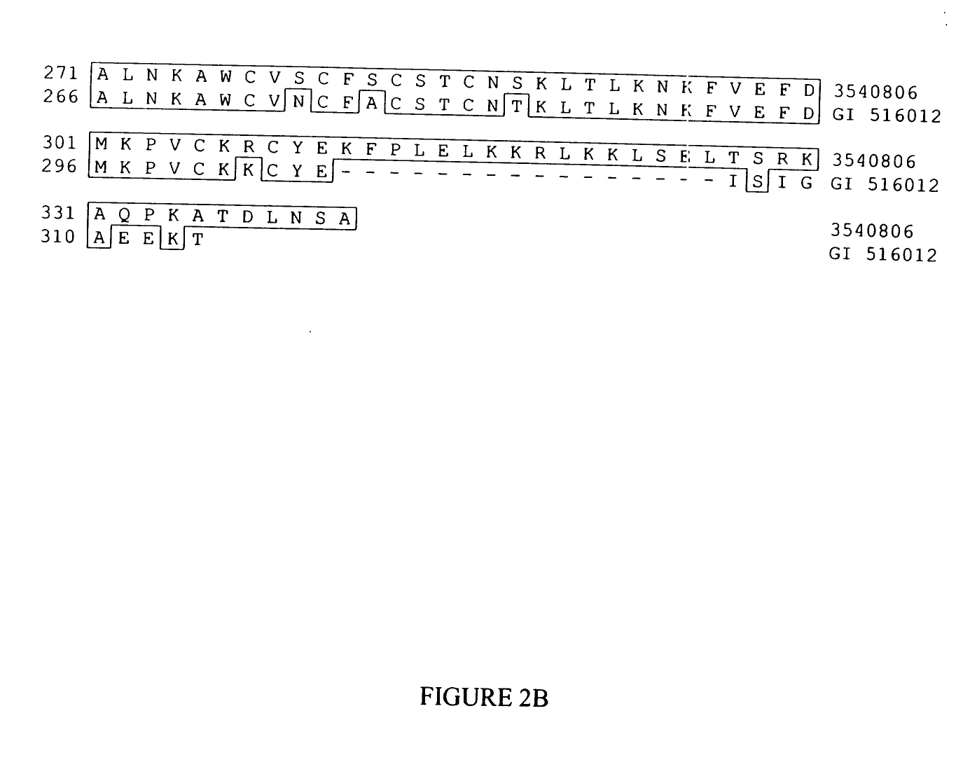

In one embodiment, the invention encompasses a protein having the amino acid sequence of SEQ ID NO:1, as shown in FIGS. 1A-1E. PINCH-PH is 341 amino acids in length and has two LIM domain signature sequences, from C₁₅ through F₅₀ and from C₇₆ through L₁₀₉. In addition, the protein has a potential amidation site at E₅₉, three potential glycosylation sites at N₅, N₁₆₅, and N₂₅₉, and a total of eight potential phosphorylation sites: a cAMP- and cGMP-dependent protein kinase phosphorylation site at S₃₂₄, three casein kinase II phosphorylation sites at S₂₃, S₃₁, and S₇₈, three protein kinase C phosphorylation sites at T₂₉₁, T₃₂₇, and S₃₂₈, and a tyrosine kinase phosphorylation site at Y₅₆. As shown in FIGS. 2A and 2B, PINCH-PH has chemical and structural homology with human PINCH (GI 516012; SEQ ID NO:3). In particular, PINCH-PH and human PINCH share 84% sequence identity, two LIM domain signature sequences, a potential amidation site, and two potential phosphorylation sites. Northern analysis shows the expression of the polynucleotide encoding PINCH-PH in reproductive, gastrointestinal, and nervous system libraries, at least 67% of which are immortalized or cancerous and at least 20% of which involve inflammation and the immune response. Of particular note is the expression of PINCH-PH in tumors of the prostate, spleen, uterus, bladder, ileum, colon, brain, and ganglion. The transcript image shown in EXAMPLE IV shows differential expression of the polynucleotide encoding PINCH-PH in prostatic adenocarcinoma and in Hodgkin's disease.

### Characterization and Use of the Invention

cDNA Libraries

In a particular embodiment disclosed herein, mRNA is isolated from mammalian cells and tissues using methods which are well known to those skilled in the art and used to prepare the cDNA libraries. The Incyte cDNAs were isolated from mammalian cDNA libraries prepared as described in the EXAMPLES. The consensus sequences are chemically and/or electronically assembled from fragments including Incyte cDNAs and extension and/or shotgun sequences using computer programs such as PHRAP (P Green, University of Washington, Seattle Wash.), and AUTOASSEMBLER application (Applied Biosystems (ABI), Foster City Calif.). After verification of the 5′ and 3′ sequence, at least one representative polynucleotide which encodes PINCH-PH is designated a reagent.

Sequencing

Methods for sequencing nucleic acids are well known in the art and may be used to practice any of the embodiments of the invention. These methods employ enzymes such as the Klenow fragment of DNA polymerase I, SEQUENASE, Taq DNA polymerase and thermostable T7 DNA polymerase (Amersham Pharmacia Biotech (APB), Piscataway N.J.), or combinations of polymerases and proofreading exonucleases such as those found in the ELONGASE amplification system (Invitrogen, Carlsbad Calif.). Preferably, sequence preparation is automated with machines such as the MICROLAB 2200 system (Hamilton, Reno Nev.) and the DNA ENGINE thermal cycler (MJ Research, Watertown Mass.). Machines commonly used for sequencing include the PRISM 3700, 377 or 373 DNA sequencing systems (ABI), the MEGABACE 1000 DNA sequencing system (APB), and the like. The sequences may be analyzed using a variety of algorithms well known in the art and described in Ausubel et al. (1997, Short Protocols in Molecular Biology, John Wiley & Sons, New York N.Y., unit 7.7) and in Meyers (1995, Molecular Biology and Biotechnology, Wiley VCH, New York N.Y., pp. 856-853).

Shotgun sequencing may also be used to complete the sequence of a particular cloned insert of interest. Shotgun strategy involves randomly breaking the original insert into segments of various sizes and cloning these fragments into vectors. The fragments are sequenced and reassembled using overlapping ends until the entire sequence of the original insert is known. Shotgun sequencing methods are well known in the art and use thermostable DNA polymerases, heat-labile DNA polymerases, and primers chosen from representative regions flanking the cDNAs of interest. Incomplete assembled sequences are inspected for identity using various algorithms or programs such as CONSED (Gordon (1998) Genome Res 8:195-202) which are well known in the art. Contaminating sequences, including vector or chimeric sequences, or deleted sequences can be removed or restored, respectively, organizing the incomplete assembled sequences into finished sequences.

Extension of a Nucleic Acid Sequence

The sequences of the invention may be extended using various PCR-based methods known in the art. For example, the XL-PCR kit (ABI), nested primers, and commercially available cDNA or genomic DNA libraries may be used to extend the nucleic acid sequence. For all PCR-based methods, primers may be designed using commercially available primer analysis software to be about 22 to 30 nucleotides in length, to have a GC content of about 50% or more, and to anneal to a target molecule at temperatures from about 55C to about 68C. When extending a sequence to recover regulatory elements, it is preferable to use genomic, rather than cDNA libraries.

Hybridization

The polynucleotide and fragments thereof can be used in hybridization technologies for various purposes. A probe may be designed or derived from unique regions such as the 5′ regulatory region or from a nonconserved region (i.e., 5′ or 3′ of the nucleotides encoding the conserved catalytic domain of the protein) and used in protocols to identify naturally occurring molecules encoding the PINCH-PH, allelic variants, or related molecules. The probe may be DNA or RNA, may be single-stranded, and should have at least 50% sequence identity to any of the nucleic acid sequences, SEQ ID NOs:2-?. Hybridization probes may be produced using oligolabeling, nick translation, end-labeling, or PCR amplification in the presence of a reporter molecule. A vector containing the polynucleotide or a fragment thereof may be used to produce an mRNA probe in vitro by addition of an RNA polymerase and labeled nucleotides. These procedures may be conducted using commercially available kits such as those provided by APB.

The stringency of hybridization is determined by G+C content of the probe, salt concentration, and temperature. In particular, stringency can be increased by reducing the concentration of salt or raising the hybridization temperature. Hybridization can be performed at low stringency with buffers, such as 5×SSC with 1% sodium dodecyl sulfate (SDS) at 60C, which permits the formation of a hybridization complex between nucleic acid sequences that contain some mismatches. Subsequent washes are performed at higher stringency with buffers such as 0.2×SSC with 0.1% SDS at either 45C (medium stringency) or 68C (high stringency). At high stringency, hybridization complexes will remain stable only where the nucleic acids are completely complementary. In some membrane-based hybridizations, preferably 35% or most preferably 50%, formamide can be added to the hybridization solution to reduce the temperature at which hybridization is performed, and background signals can be reduced by the use of detergents such as Sarkosyl or TRITON X-100 (Sigma-Aldrich, St. Louis Mo.) and a blocking agent such as denatured salmon sperm DNA. Selection of components and conditions for hybridization are well known to those skilled in the art and are reviewed in Ausubel (supra) and Sambrook et al. (1989) Molecular Cloning, A Laboratory Manual, Cold Spring Harbor Press, Plainview N.Y.

Arrays may be prepared and analyzed using methods well known in the art. Oligonucleotides or cDNAs may be used as hybridization probes or targets to monitor the expression level of large numbers of genes simultaneously or to identify genetic variants, mutations, and single nucleotide polymorphisms. Arrays may be used to determine gene function; to understand the genetic basis of a condition, disease, or disorder; to diagnose a condition, disease, or disorder; and to develop and monitor the activities of therapeutic agents. (See, e.g., Brennan et al. (1995) U.S. Pat. No. 5,474,796; Schena et al. (1996) Proc Natl Acad Sci 93:10614-10619; Heller et al. (1997) Proc Natl Acad Sci 94:2150-2155; and Heller et al. (1997) U.S. Pat. No. 5,605,662.)

Hybridization probes are also useful in mapping the naturally occurring genomic sequence. The probes may be hybridized to a particular chromosome, a specific region of a chromosome, or an artificial chromosome construction. Such constructions include human artificial chromosomes (HAC), yeast artificial chromosomes (YAC), bacterial artificial chromosomes (BAC), bacterial P1 constructions, or the cDNAs of libraries made from single chromosomes.

Quantitative PCR (TAQMAN, ABI)

Quantitative real-time PCR (QPCR) is a method for quantifying a nucleic acid molecule based on detection of a fluorescent signal produced during PCR amplification (Gibson et al. (1996) Genome Res 6:995-1001; Heid et al. (1996) Genome Res 6:986-994). Amplification is carried out on machines such as the PRISM 7700 detection system which consists of a 96-well thermal cycler connected to a laser and charge-coupled device (CCD) optics system. To perform QPCR, a PCR reaction is carried out in the presence of a doubly labeled “TAQMAN” probe. The probe, which is designed to anneal between the standard forward and reverse PCR primers, is labeled at the 5′ end by a flourogenic reporter dye such as 6-carboxyfluorescein (6-FAM) and at the 3′ end by a quencher molecule such as 6-carboxy-tetramethyl-rhodamine (TAMRA). As long as the probe is intact, the 3′ quencher extinguishes fluorescence by the 5′ reporter. However, during each primer extension cycle, the annealed probe is degraded as a result of the intrinsic 5′ to 3′ nuclease activity of Taq polymerase (Holland et al. (1991) Proc Natl Acad Sci 88:7276-7280). This degradation separates the reporter from the quencher, and fluorescence is detected every few seconds by the CCD. The higher the starting copy number of the nucleic acid , the sooner a significant increase in fluorescence is observed. A cycle threshold (C_{T}) value, representing the cycle number at which the PCR product crosses a fixed threshold of detection is determined by the instrument software. The C_{T} is inversely proportional to the copy number of the template and can therefore be used to calculate either the relative or absolute initial concentration of the nucleic acid molecule in the sample. The relative concentration of two different molecules can be calculated by determining their respective C_{T} values (comparative C_{T} method). Alternatively, the absolute concentration of the nucleic acid molecule can be calculated by constructing a standard curve using a housekeeping molecule of known concentration. The process of calculating C_{T}s, preparing a standard curve, and determining starting copy number is performed by the SEQUENCE DETECTOR 1.7 software (ABI).

Expression

Any one of a multitude of polynucleotides encoding PINCH-PH may be cloned into a vector and used to express the protein, or portions thereof, in host cells. The nucleic acid sequence can be engineered by such methods as DNA shuffling (U.S. Pat. No. 5,830,721) and site-directed mutagenesis to create new restriction sites, alter glycosylation patterns, change codon preference to increase expression in a particular host, produce splice variants, extend half-life, and the like. The expression vector may contain transcriptional and translational control elements (promoters, enhancers, specific initiation signals, and polyadenylated 3′ sequence) from various sources which have been selected for their efficiency in a particular host. The vector, polynucleotide, and regulatory elements are combined using in vitro recombinant DNA techniques, synthetic techniques, and/or in vivo genetic recombination techniques well known in the art and described in Sambrook (supra, ch. 4, 8, 16 and 17).

A variety of host systems may be transformed with an expression vector. These include, but are not limited to, bacteria transformed with recombinant bacteriophage, plasmid, or cosmid DNA expression vectors; yeast transformed with yeast expression vectors; insect cell systems transformed with baculovirus expression vectors; plant cell systems transformed with expression vectors containing viral and/or bacterial elements, or animal cell systems (Ausubel supra, unit 16). For example, an adenovirus transcription/translation complex may be utilized in mammalian cells. After sequences are ligated into the E1 or E3 region of the viral genome, the infective virus is used to transform and express the protein in host cells. The Rous sarcoma virus enhancer or SV40 or EBV-based vectors may also be used for high-level protein expression.

Routine cloning, subcloning, and propagation of nucleic acid sequences can be achieved using the multifunctional pBLUESCRIPT vector (Stratagene, La Jolla Calif.) or pSPORT1 plasmid (Invitrogen). Introduction of a nucleic acid sequence into the multiple cloning site of these vectors disrupts the lacZ gene and allows colorimetric screening for transformed bacteria. In addition, these vectors may be useful for in vitro transcription, dideoxy sequencing, single strand rescue with helper phage, and creation of nested deletions in the cloned sequence.

For long term production of recombinant proteins, the vector can be stably transformed into cell lines along with a selectable or visible marker gene on the same or on a separate vector. After transformation, cells are allowed to grow for about 1 to 2 days in enriched media and then are transferred to selective media. Selectable markers, antimetabolite, antibiotic, or herbicide resistance genes, confer resistance to the relevant selective agent and allow growth and recovery of cells which successfully express the introduced sequences. Resistant clones identified either by survival on selective media or by the expression of visible markers may be propagated using culture techniques. Visible markers are also used to estimate the amount of protein expressed by the introduced genes. Verification that the host cell contains the desired polynucleotide is based on DNA-DNA or DNA-RNA hybridizations or PCR amplification techniques.

The host cell may be chosen for its ability to modify a recombinant protein in a desired fashion. Such modifications include acetylation, carboxylation, glycosylation, phosphorylation, lipidation, acylation and the like. Post-translational processing which cleaves a “prepro” form may also be used to specify protein targeting, folding, and/or activity. Different host cells available from the ATCC (Manassas Va.) which have specific cellular machinery and characteristic mechanisms for post-translational activities may be chosen to ensure the correct modification and processing of the recombinant protein.

Recovery of Proteins from Cell Culture

Heterologous moieties engineered into a vector for ease of purification include glutathione S-transferase (GST), 6×His, FLAG, MYC, and the like. GST and 6-His are purified using commercially available affinity matrices such as immobilized glutathione and metal-chelate resins, respectively. FLAG and MYC are purified using commercially available monoclonal and polyclonal antibodies. For ease of separation following purification, a sequence encoding a proteolytic cleavage site may be part of the vector located between the protein and the heterologous moiety. Methods for recombinant protein expression and purification are discussed in Ausubel (supra, unit 16) and are commercially available.

Protein Identification

Several techniques have been developed which permit rapid identification of proteins using high performance liquid chromatography and mass spectrometry. Beginning with a sample containing proteins, the major steps involved are: 1) proteins are separated using two-dimensional gel electrophoresis (2-DE), 2) selected proteins are excised from the gel and digested with a protease to produce a set of peptides; and 3) the peptides are subjected to mass spectral (MS) analysis to derive peptide ion mass and spectral pattern information. The MS information is used to identify the protein by comparing it with information in a protein database (Shevenko et al.(1996) Proc Natl Acad Sci 93:14440-14445). A more detailed description follows.

Proteins are separated by 2DE employing isoelectric focusing (IEF) in the first dimension followed by SDS-PAGE in the second dimension. For IEF, an immobilized pH gradient strip is useful to increase reproducibility and resolution of the separation. Alternative techniques may be used to improve resolution of very basic, hydrophobic, or high molecular weight proteins. The separated proteins are detected using a stain or dye such as silver stain, Coomassie blue, or spyro red (Molecular Bioprobes, Eugene Oreg.) that is compatible with mass spectrometry Gels may be blotted onto a PVDF membrane for western analysis and optically scanned using a STORM scanner (APB) to produce a computer-readable output which is analyzed by pattern recognition software such as MELANIE (GeneBio, Geneva, Switzerland). The software annotates individual spots by assigning a unique identifier and calculating their respective x,y coordinates, molecular masses, isoelectric points, and signal intensity. Individual spots of interest, such as those representing differentially expressed proteins, are excised and proteolytically digested with a site-specific protease such as trypsin or chymotrypsin, singly or in combination, to generate a set of small peptides, preferably in the range of 1-2 kDa. Prior to digestion, samples may be treated with reducing and alkylating agents, and following digestion, the peptides are then separated by liquid chromatography or capillary electrophoresis and analyzed using MS.

MS converts components of a sample into gaseous ions, separates the ions based on their mass-to-charge ratio, and determines relative abundance. For peptide mass fingerprinting analysis, a mass spectrometer of the MALDI-TOF (Matrix Assisted Laser Desorption/Ionization-Time of Flight), ESI (Electrospray Ionization), and TOF-TOF (Time of Flight/Time of Flight) machines are used to determine a set of highly accurate peptide masses. Using analytical programs, such as TURBOSEQUEST software (Finnigan, San Jose Calif.), the MS data is compared against a database of theoretical MS data derived from known or predicted proteins. A minimum match of three peptide masses is usually required for reliable protein identification. If additional information is needed for identification, Tandem-MS may be used to derive information about individual peptides. In tandem-MS, a first stage of MS is performed to determine individual peptide masses. Then selected peptide ions are subjected to fragmentation using a technique such as collision induced dissociation (CID) to produce an ion series. The resulting fragmentation ions are analyzed in a second round of MS, and their spectral pattern may be used to determine a short stretch of amino acid sequence (Dancik et al. (1999) J Comput Biol 6:327-342).

Assuming the protein is represented in the database, a combination of peptide mass and fragmentation data, together with the calculated MW and pI of the protein, will usually yield an unambiguous identification. If no match is found, protein sequence can be obtained using direct chemical sequencing procedures well known in the art (cf Creighton (1984) Proteins, Structures and Molecular Properties, W H Freeman, New York N.Y.).

Chemical Synthesis of Peptides

Proteins or portions thereof may be produced not only by recombinant methods, but also by using chemical methods well known in the art. Solid phase peptide synthesis may be carried out in a batchwise or continuous flow process which sequentially adds α-amino- and side chain-protected amino acid residues to an insoluble polymeric support via a linker group. A linker group such as methylamine-derivatized polyethylene glycol is attached to poly(styrene-co-divinylbenzene) to form the support resin. The amino acid residues are N-α-protected by acid labile Boc (t-butyloxycarbonyl) or base-labile Fmoc (9-fluorenylmethoxycarbonyl). The carboxyl group of the protected amino acid is coupled to the amine of the linker group to anchor the residue to the solid phase support resin. Trifluoroacetic acid or piperidine are used to remove the protecting group in the case of Boc or Fmoc, respectively. Each additional amino acid is added to the anchored residue using a coupling agent or pre-activated amino acid derivative, and the resin is washed. The full length peptide is synthesized by sequential deprotection, coupling of derivatized amino acids, and washing with dichloromethane and/or N, N-dimethylformamide. The peptide is cleaved between the peptide carboxy terminus and the linker group to yield a peptide acid or amide. (Novabiochem 1997/98 Catalog and Peptide Synthesis Handbook, San Diego Calif. pp. S1-S20). Automated synthesis may also be carried out on machines such as the 431A peptide synthesizer (ABI). A protein or portion thereof may be purified by preparative high performance liquid chromatography and its composition confirmed by amino acid analysis or by sequencing (Creighton (1984) Proteins, Structures and Molecular Properties, W H Freeman, New York N.Y.).

Antibodies

Antibodies, or immunoglobulins (Ig), are components of immune response expressed on the surface of or secreted into the circulation by B cells. The prototypical antibody is a tetramer composed of two identical heavy polypeptide chains (H-chains) and two identical light polypeptide chains (L-chains) interlinked by disulfide bonds which binds and neutralizes foreign antigens. Based on their H-chain, antibodies are classified as IgA, IgD, IgE, IgG or IgM. The most common class, IgG, is tetrameric while other classes are variants or multimers of the basic structure.

Antibodies are described in terms of their two main functional domains. Antigen recognition is mediated by the Fab (antigen binding fragment) region of the antibody, while effector functions are mediated by the Fc (crystallizable fragment) region. The binding of antibody to antigen triggers destruction of the antigen by phagocytic white blood cells such as macrophages and neutrophils. These cells express surface Fc receptors that specifically bind to the Fc region of the antibody and allow the phagocytic cells to destroy antibody-bound antigen. Fc receptors are single-pass transmembrane glycoproteins containing about 350 amino acids whose extracellular portion typically contains two or three Ig domains (Sears et al. (1990) J Immunol 144:371-378).

Preparation and Screening of Antibodies

Various hosts including mice, rats, rabbits, goats, llamas, camels, and human cell lines may be immunized by injection with an antigenic determinant. Adjuvants such as Freund's, mineral gels, and surface active substances such as lysolecithin, pluronic polyols, polyanions, peptides, oil emulsions, keyhole limpet hemacyanin (KLH; Sigma-Aldrich, St. Louis Mo.), and dinitrophenol may be used to increase immunological response. In humans, BCG (bacilli Calmette-Guerin) and Corynebacterium parvum are preferable. The antigenic determinant may be an oligopeptide, peptide, or protein. When the amount of antigenic determinant allows immunization to be repeated, specific polyclonal antibody with high affinity can be obtained (Klinman and Press (1975) Transplant Rev 24:41-83). Oligopepetides which may contain between about five and about fifteen amino acids identical to a portion of the endogenous protein may be fused with proteins such as KLH in order to produce antibodies to the chimeric molecule.

Monoclonal antibodies may be prepared using any technique which provides for the production of antibodies by continuous cell lines in culture. These include the hybridoma technique, the human B-cell hybridoma technique, and the EBV-hybridoma technique (Kohler et al (1975) Nature 256:495-497; Kozbor et al (1985) J Immunol Methods 81:31-42; Cote et al (1983) Proc Natl Acad Sci 80:2026-2030; and Cole et al (1984) Mol Cell Biol 62:109-120).

Chimeric antibodies may be produced by techniques such as splicing of mouse antibody genes to human antibody genes to obtain a molecule with appropriate antigen specificity and biological activity (Morrison et al. (1984) Proc Natl Acad Sci 81:6851-6855; Neuberger et al. (1984) Nature 312:604-608; and Takeda et al. (1985) Nature 314:452-454). Alternatively, techniques described for antibody production may be adapted, using methods known in the art, to produce specific, single chain antibodies. Antibodies with related specificity, but of distinct idiotypic composition, may be generated by chain shuffling from random combinatorial immunoglobulin libraries (Burton (1991) Proc Natl Acad Sci 88:10134-10137). Antibody fragments which contain specific binding sites for an antigenic determinant may also be produced. For example, such fragments include, but are not limited to, F(ab′)2 fragments produced by pepsin digestion of the antibody molecule and Fab fragments generated by reducing the disulfide bridges of the F(ab′)2 fragments. Alternatively, Fab expression libraries may be constructed to allow rapid and easy identification of monoclonal Fab fragments with the desired specificity (Huse et al. (1989) Science 246:1275-1281).

Antibodies may also be produced by inducing production in the lymphocyte population or by screening immunoglobulin libraries or panels of highly specific binding reagents as disclosed in Orlandi et al. (1989; Proc Natl Acad Sci 86:3833-3837) or Winter et al. (1991; Nature 349:293-299). A protein may be used in screening assays of phagemid or B-lymphocyte immunoglobulin libraries to identify antibodies having a desired specificity. Numerous protocols for competitive binding or immunoassays using either polyclonal or monoclonal antibodies with established specificities are well known in the art.

Antibody Specificity

Various methods such as Scatchard analysis combined with radioimmunoassay techniques may be used to assess the affinity of particular antibodies for a protein. Affinity is expressed as an association constant, Kₐ, which is defined as the molar concentration of protein-antibody complex divided by the molar concentrations of free antigen and free antibody under equilibrium conditions. The Kₐ determined for a preparation of polyclonal antibodies, which are heterogeneous in their affinities for multiple antigenic determinants, represents the average affinity, or avidity, of the antibodies. The Kₐ determined for a preparation of monoclonal antibodies, which are specific for a particular antigenic determinant, represents a true measure of affinity. High-affinity antibody preparations with Kₐ ranging from about 10⁹ to 10¹² L/mole are preferred for use in immunoassays in which the protein-antibody complex must withstand rigorous manipulations. Low-affinity antibody preparations with Kₐ ranging from about 10⁶ to 10⁷ L/mole are preferred for use in immunopurification and similar procedures which ultimately require dissociation of the protein, preferably in active form, from the antibody (Catty (1988) Antibodies, Volume I: A Practical Approach, IRL Press, Washington D.C.; Liddell and Cryer (1991) A Practical Guide to Monoclonal Antibodies, John Wiley & Sons, New York N.Y.).

The titer and avidity of polyclonal antibody preparations may be further evaluated to determine the quality and suitability of such preparations for certain downstream applications. For example, a polyclonal antibody preparation containing about 5-10 mg specific antibody/ml, is generally employed in procedures requiring precipitation of protein-antibody complexes. Procedures for making antibodies, evaluating antibody specificity, titer, and avidity, and guidelines for antibody quality and usage in various applications, are widely available (Catty (supra); Ausubel (supra) pp. 11.1-11.31).

Immunological Assays

Immunological methods for detecting and measuring complex formation as a measure of protein expression using either specific polyclonal or monoclonal antibodies are known in the art. Examples of such techniques include enzyme-linked immunosorbent assays (ELISAs), radioimmunoassay (RIAs), fluorescence-activated cell sorting (FACS) and antibody arrays. Such immunoassays typically involve the measurement of complex formation between the protein and its specific antibody. A two-site, monoclonal-based immunoassays utilizing antibodies reactive to two non-interfering epitopes is preferred, but a competitive binding assay may be employed (Pound (1998) Immunochemical Protocols, Humana Press, Totowa N.J.).

These methods are also useful for diagnosing diseases that show differential protein expression. Normal or standard values for protein expression are established by combining body fluids or cell extracts taken from a normal mammalian or human subject with specific antibodies to a protein under conditions for complex formation. Standard values for complex formation in normal and diseased tissues are established by various methods, often photometric means. Then complex formation as it is expressed in a subject sample is compared with the standard values. Deviation from the normal standard and toward the diseased standard provides parameters for disease diagnosis or prognosis while deviation away from the diseased and toward the normal standard may be used to evaluate treatment efficacy.

Labeling of Molecules for Assay

A wide variety of reporter molecules and conjugation techniques are known by those skilled in the art and may be used in various nucleic acid, amino acid, and antibody assays. Synthesis of labeled molecules may be achieved using commercially available kits (Promega, Madison Wis.) for incorporation of a labeled nucleotide such as ³²P-dCTP (APB), Cy3-dCTP or Cy5-dCTP (Qiagen-Operon, Alameda Calif.), or amino acid such as ³⁵S-methionine (APB). Nucleotides and amino acids may be directly labeled with a variety of substances including fluorescent, chemiluminescent, or chromogenic agents, and the like, by chemical conjugation to amines, thiols and other groups present in the molecules using reagents such as BIODIPY or FITC (Molecular Probes, Eugene Oreg.).

### DIAGNOSTICS

Nucleic Acid Assays

The polynucleotides, fragments, oligonucleotides, complementary RNA and DNA molecules, and peptide nucleic acids and may be used to detect and quantify differential gene expression for diagnosis of a disorder. The diagnostic assay may use hybridization or amplification technology to compare gene expression in a biological sample from a patient to standard samples in order to detect differential gene expression. Qualitative or quantitative methods for this comparison are well known in the art.

For example, the polynucleotide or probe may be labeled by standard methods and added to a biological sample from a patient under conditions for the formation of hybridization complexes. After an incubation period, the sample is washed and the amount of label (or signal) associated with hybridization complexes, is quantified and compared with a standard value. If complex formation in the patient sample is significantly altered (higher or lower) in comparison to either a normal or disease standard, then differential expression indicates the presence of a disorder.

Protein Assays

Detection and quantification of a protein using either labeled amino acids or specific polyclonal or monoclonal antibodies are known in the art. Examples of such techniques include two-dimensional polyacrylamide gel electrophoresis, enzyme-linked immunosorbent assays (ELISAs), radioimmunoassays (RIAs), and fluorescence activated cell sorting (FACS). These assays and their quantitation against purifed, labeled standards are well known in the art (Ausubel, supra, unit 10.1-10.6).

Recently, antibody arrays have allowed the development of techniques for high-throughput screening of recombinant antibodies. Such methods use robots to pick and grid bacteria containing antibody genes, and a filter-based ELISA to screen and identify clones that express antibody fragments. Because liquid handling is eliminated and the clones are arrayed from master stocks, the same antibodies can be spotted multiple times and screened against multiple antigens simultaneously. Antibody arrays are highly useful in the identification of differentially expressed proteins (See de Wildt et al. (2000) Nature Biotechnol 18:989-94.)

In order to provide standards for establishing differential expression, normal and disease expression profiles are established. This is accomplished by combining a sample taken from normal subjects, either animal or human, with the polynucleotide encoding PINCH-PH under conditions for hybridization to occur. Standard hybridization complexes may be quantified by comparing the values obtained using normal subjects with values from an experiment in which a known amount of a purified sequence is used. Standard values obtained in this manner may be compared with values obtained from samples from patients who were diagnosed with a particular condition, disease, or disorder. Deviation from standard values toward those associated with a particular disorder is used to diagnose that disorder.

Efficacy

Both nucleic acid and protein assays may also be used to evaluate the efficacy of a particular therapeutic treatment regimen in animal studies or in clinical trials or to monitor the treatment of an individual patient. Once the presence of a condition is established and a treatment protocol is initiated, diagnostic assays may be repeated on a regular basis to determine if the level of expression in the patient begins to approximate that which is observed in a normal subject. The results obtained from successive assays may be used to show the efficacy of treatment over a period ranging from several days to years. Disorders associated with differential expression of PINCH-PH include cancers such as adenocarcinoma, leukemia, lymphoma, melanoma, myeloma, sarcoma, teratocarcinoma, and, in particular, cancers of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus, and reproductive disorders, such as disorders of prolactin production; infertility including tubal disease, ovulatory defects, and endometriosis; disruption of the estrous or menstrual cycles, polycystic ovary syndrome, endometrial and ovarian tumors; cancer of the male or female breast, fibrocystic breast disease, and galactorrhea; disruptions of spermatogenesis, cancer of the testis or prostate, and prostatitis. When used in a tissue specific and clinically relevant manner, differential expression of PINCH-PH as detected using nucleic acid or protein assays is diagnostic of cancer and reproductive disorders, particularly prostatic adenocarcinoma and Hodgkins disease.

### THERAPEUTICS

Chemical and structural similarity, in particular the five LIM binding domains, between PINCH-PH (SEQ ID NO:1) and the PINCH protein (GI516012; SEQ ID NO:3) are shown in FIG. 2. In addition, differential expression of PINCH-PH is highly associated with cancer and with reproductive disorders. The transcript images of Example IV also shown that differential expression of PINCH-PH plays a role in prostatic adenocarcinoma and Hodgkin's disease.

In one embodiment, when decreased expression of activity of the protein is desired, an inhibitor, antagonist, antibody and the like or a pharmaceutical agent containing one or more of these molecules may be delivered. Such delivery may be effected by methods well known in the art and may include delivery by an antibody specifically targeted to the protein. Neutralizing antibodies which inhibit dimer formation are generally preferred for therapeutic use.

In another embodiment, when increased expression or activity of the protein is desired, the protein, an agonist, an enhancer and the like or a pharmaceutical agent containing one or more of these molecules may be delivered. Such delivery may be effected by methods well known in the art and may include delivery of a pharmaceutical agent by an antibody specifically targeted to the protein.

Any of the polynucleotides, complementary molecules, or fragments thereof, proteins or portions thereof, vectors delivering these nucleic acid molecules or expressing the proteins, and their ligands may be administered in combination with other therapeutic agents. Selection of the agents for use in combination therapy may be made by one of ordinary skill in the art according to conventional pharmaceutical principles. A combination of therapeutic agents may act synergistically to affect treatment of a particular disorder at a lower dosage of each agent.

Modification of Gene Expression Using Nucleic Acids

Gene expression may be modified by designing complementary or antisense molecules (DNA, RNA, or PNA) to the control, 5′, 3′, or other regulatory regions of the gene encoding PINCH-PH. Oligonucleotides designed to inhibit transcription initiation are preferred. Similarly, inhibition can be achieved using triple helix base-pairing which inhibits the binding of polymerases, transcription factors, or regulatory molecules (Gee et al. In: Huber and Carr (1994) Molecular and Immunologic Approaches, Futura Publishing, Mt. Kisco N.Y., pp. 163-177). A complementary molecule may also be designed to block translation by preventing binding between ribosomes and mRNA. In one alternative, a library or plurality of polynucleotides may be screened to identify those which specifically bind a regulatory, nontranslated sequence.

Ribozymes, enzymatic RNA molecules, may also be used to catalyze the specific cleavage of RNA. The mechanism of ribozyme action involves sequence-specific hybridization of the ribozyme molecule to complementary target RNA followed by endonucleolytic cleavage at sites such as GUA, GUU, and GUC. Once such sites are identified, an oligonucleotide with the same sequence may be evaluated for secondary structural features which would render the oligonucleotide inoperable. The suitability of candidate targets may also be evaluated by testing their hybridization with complementary oligonucleotides using ribonuclease protection assays.

Complementary nucleic acids and ribozymes of the invention may be prepared via recombinant expression, in vitro or in vivo, or using solid phase phosphoramidite chemical synthesis. In addition, RNA molecules may be modified to increase intracellular stability and half-life by addition of flanking sequences at the 5′ and/or 3′ ends of the molecule or by the use of phosphorothioate or 2′ O-methyl rather than phosphodiesterase linkages within the backbone of the molecule. Modification is inherent in the production of PNAs and can be extended to other nucleic acid molecules. Either the inclusion of nontraditional bases such as inosine, queosine, and wybutosine, or the modification of adenine, cytidine, guanine, thymine, and uridine with acetyl-, methyl-, thio- groups renders the molecule less available to endogenous endonucleases.

Nucleic Acid Therapeutics

The polynucleotides of the invention can be used in gene therapy. The polynucleotides can be delivered ex vivo to target cells, such as the cells of bone marrow. Once stable integration and transcription and or translation are confirmed, the bone marrow may be reintroduced into the subject. Expression of the protein encoded by the polynucleotide may lessen or even correct a disorder associated with mutation of a normal sequence, reduction or loss of an endogenous target protein, or overepression of an endogenous or mutant protein. Alternatively, polynucleotides may be delivered in vivo using vectors such as retrovirus, adenovirus, adeno-associated virus, herpes simplex virus, and bacterial plasmids. Non-viral methods of gene delivery include cationic liposomes, polylysine conjugates, artificial viral envelopes, and direct injection of DNA (Anderson (1998) Nature 392:25-30; Dachs et al. (1997) Oncol Res 9:313-325; Chu et al. (1998) J Mol Med 76(3-4):184-192; Weiss et al. (1999) Cell Mol Life Sci 55(3):334-358; Agrawal (1996) Antisense Therapeutics, Humana Press, Totowa N.J.; and August et al. (1997) Gene Therapy (Advances in Pharmacology, Vol. 40, Academic Press, San Diego Calif.).

Screening and Purification Assays

The polynucleotide encoding PINCH-PH may be used to screen a library or a plurality of molecules or compounds for specific binding affinity. The libraries may be cDNA molecules, RNA molecules, peptide nucleic acids, peptides, proteins such as transcription factors, enhancers, or repressors, and other ligands which regulate the activity, replication, transcription, or translation of the endogenous gene. The assay involves combining a polynucleotide with a library or plurality of molecules or compounds under conditions allowing specific binding, and detecting specific binding to identify at least one molecule which specifically binds the single-stranded or double-stranded molecule.

In one embodiment, the polynucleotide of the invention may be incubated with a plurality of purified molecules or compounds and binding activity determined by methods well known in the art, e.g., a gel-retardation assay (U.S. Pat. No. 6,010,849) or a reticulocyte lysate transcriptional assay. In another embodiment, the polynucleotide may be incubated with nuclear extracts from biopsied and/or cultured cells and tissues. Specific binding between the polynucleotide and a molecule or compound in the nuclear extract is initially determined by gel shift assay and may be later confirmed by recovering and raising antibodies against that molecule or compound. When these antibodies are added into the assay, they cause a supershift in the gel-retardation assay.

In another embodiment, the polynucleotide may be used to purify a molecule or compound using affinity chromatography methods well known in the art. In one embodiment, the polynucleotide is chemically reacted with cyanogen bromide groups on a polymeric resin or gel. Then a sample is passed over and reacts with or binds to the polynucleotide. The molecule or compound which is bound to the polynucleotide may be released from it by increasing the salt concentration of the flow-through medium and collected.

In a further embodiment, the protein or a portion thereof may be used to purify a ligand from a sample. A method for using a protein or a portion thereof to purify a ligand would involve combining the protein or a portion thereof with a sample under conditions to allow specific binding, detecting specific binding between the protein and ligand, recovering the bound protein, and using a chaotropic agent to separate the protein from the purified ligand.

In a preferred embodiment, PINCH-PH may be used to screen a plurality of molecules or compounds in any of a variety of screening assays. The portion of the protein employed in such screening may be free in solution, affixed to an abiotic or biotic substrate (e.g. borne on a cell surface), or located intracellularly. For example, in one method, viable or fixed prokaryotic host cells that are stably transformed with recombinant nucleic acids that have expressed and positioned a peptide on their cell surface can be used in screening assays. The cells are screened against a plurality or libraries of ligands, and the specificity of binding or formation of complexes between the expressed protein and the ligand can be measured. Depending on the particular kind of molecules or compounds being screened, the assay may be used to identify DNA molecules, RNA molecules, peptide nucleic acids, peptides, proteins, mimetics, agonists, antagonists, antibodies, immunoglobulins, inhibitors, and drugs or any other ligand, which specifically binds the protein.

In one aspect, this invention contemplates a method for high throughput screening using very small assay volumes and very small amounts of test compound as described in U.S. Pat. No. 5,876,946, incorporated herein by reference. This method is used to screen large numbers of molecules and compounds via specific binding. In another aspect, this invention also contemplates the use of competitive drug screening assays in which neutralizing antibodies capable of binding the protein specifically compete with a test compound capable of binding to the protein. Molecules or compounds identified by screening may be used in a mammalian model system to evaluate their toxicity, diagnostic, or therapeutic potential.

Pharmaceutical Compositions

Pharmaceutical compositions may be formulated and administered, to a subject in need of such treatment, to attain a therapeutic effect. Such compositions contain the instant protein, agonists, antibodies specifically binding the protein, antagonists, inhibitors, or mimetics of the protein. Compositions may be manufactured by conventional means such as mixing, dissolving, granulating, dragee-making, levigating, emulsifying, encapsulating, entrapping, or lyophilizing. The composition may be provided as a salt, formed with acids such as hydrochloric, sulfuric, acetic, lactic, tartaric, malic, and succinic, or as a lyophilized powder which may be combined with a sterile buffer such as saline, dextrose, or water. These compositions may include auxiliaries or excipients which facilitate processing of the active compounds.

Auxiliaries and excipients may include coatings, fillers or binders including sugars such as lactose, sucrose, mannitol, glycerol, or sorbitol; starches from corn, wheat, rice, or potato; proteins such as albumin, gelatin and collagen; cellulose in the form of hydroxypropymethyl-cellulose, methyl cellulose, or sodium carboxymethylcellulose; gums including arabic and tragacanth; lubricants such as magnesium stearate or talc; disintegrating or solubilizing agents such as the, agar, alginic acid, sodium alginate or cross-linked polyvinyl pyrrolidone; stabilizers such as carbopol gel, polyethylene glycol, or titanium dioxide; and dyestuffs or pigments added for identify the product or to characterize the quantity of active compound or dosage.

These compositions may be administered by any number of routes including oral, intravenous, intramuscular, intra-arterial, intramedullary, intrathecal, intraventricular, transdermal, subcutaneous, intraperitoneal, intranasal, enteral, topical, sublingual, or rectal.

The route of administration and dosage will determine formulation; for example, oral administration may be accomplished using tablets, pills, dragees, capsules, liquids, gels, syrups, slurries, or suspensions; parenteral administration may be formulated in aqueous, physiologically compatible buffers such as Hanks' solution, Ringer's solution, or physiologically buffered saline. Suspensions for injection may be aqueous, containing viscous additives such as sodium carboxymethyl cellulose or dextran to increase the viscosity, or oily, containing lipophilic solvents such as sesame oil or synthetic fatty acid esters such as ethyl oleate or triglycerides, or liposomes. Penetrants well known in the art are used for topical or nasal administration.

Toxicity and Therapeutic Efficacy

A therapeutically effective dose refers to the amount of active ingredient which ameliorates symptoms or condition. For any compound, a therapeutically effective dose can be estimated from cell culture assays using normal and neoplastic cells or in animal models. Therapeutic efficacy, toxicity, concentration range, and route of administration may be determined by standard pharmaceutical procedures using experimental animals.

The therapeutic index is the dose ratio between therapeutic and toxic effects—LD50 (the dose lethal to 50% of the population)/ED50 (the dose therapeutically effective in 50% of the population)—and large therapeutic indices are preferred. Dosage is within a range of circulating concentrations, includes an ED50 with little or no toxicity, and varies depending upon the composition, method of delivery, sensitivity of the patient, and route of administration. Exact dosage will be determined by the practitioner in light of factors related to the subject in need of the treatment.

Dosage and administration are adjusted to provide active moiety that maintains therapeutic effect. Factors for adjustment include the severity of the disease state, general health of the subject, age, weight, and gender of the subject, diet, time and frequency of administration, drug combination(s), reaction sensitivities and tolerance/response to therapy. Long-acting pharmaceutical compositions may be administered every 3 to 4 days, every week, or once every two weeks depending on half-life and clearance rate of the particular composition.

Normal dosage amounts may vary from 0.1 μg, up to a total dose of about 1 g, depending upon the route of administration. The dosage of a particular composition may be lower when administered to a patient in combination with other agents, drugs, or hormones. Guidance as to particular dosages and methods of delivery is provided in the pharmaceutical literature and generally available to practitioners. Further details on techniques for formulation and administration may be found in the latest edition of Remington's Pharmaceutical Sciences (Mack Publishing, Easton Pa.).

### Model Systems

Animal models may be used as bioassays where they exhibit a phenotypic response similar to that of humans and where exposure conditions are relevant to human exposures. Mammals are the most common models, and most infectious agent, cancer, drug, and toxicity studies are performed on rodents such as rats or mice because of low cost, availability, lifespan, reproductive potential, and abundant reference literature. Inbred and outbred rodent strains provide a convenient model for investigation of the physiological consequences of under- or over-expression of genes of interest and for the development of methods for diagnosis and treatment of diseases. A mammal inbred to over-express a particular gene (for example, secreted in milk) may also serve as a convenient source of the protein expressed by that gene.

Toxicology

Toxicology is the study of the effects of agents on living systems. The majority of toxicity studies are performed on rats or mice. Observation of qualitative and quantitative changes in physiology, behavior, homeostatic processes, and lethality in the rats or mice are used to generate a toxicity profile and to assess potential consequences on human health following exposure to the agent.

Genetic toxicology identifies and analyzes the effect of an agent on the rate of endogenous, spontaneous, and induced genetic mutations. Genotoxic agents usually have common chemical or physical properties that facilitate interaction with nucleic acids and are most harmful when chromosomal aberrations are transmitted to progeny. Toxicological studies may identify agents that increase the frequency of structural or functional abnormalities in the tissues of the progeny if administered to either parent before conception, to the mother during pregnancy, or to the developing organism. Mice and rats are most frequently used in these tests because their short reproductive cycle allows the production of the numbers of organisms needed to satisfy statistical requirements.

Acute toxicity tests are based on a single administration of an agent to the subject to determine the symptomology or lethality of the agent. Three experiments are conducted: 1) an initial dose-range-finding experiment, 2) an experiment to narrow the range of effective doses, and 3) a final experiment for establishing the dose-response curve.

Subchronic toxicity tests are based on the repeated administration of an agent Rat and dog are commonly used in these studies to provide data from species in different families. With the exception of carcinogenesis, there is considerable evidence that daily administration of an agent at high-dose concentrations for periods of three to four months will reveal most forms of toxicity in adult animals.

Chronic toxicity tests, with a duration of a year or more, are used to demonstrate either the absence of toxicity or the carcinogenic potential of an agent. When studies are conducted on rats, a minimum of three test groups plus one control group are used, and animals are examined and monitored at the outset and at intervals throughout the experiment.

Transgenic Animal Models

Transgenic rodents that over-express or under-express a gene of interest may be inbred and used to model human diseases or to test therapeutic or toxic agents. (See, e.g., U.S. Pat. No. 5,175,383 and U.S. Pat. No. 5,767,337.) In some cases, the introduced gene may be activated at a specific time in a specific tissue type during fetal or postnatal development. Expression of the transgene is monitored by analysis of phenotype, of tissue-specific mRNA expression, or of serum and tissue protein levels in transgenic animals before, during, and after challenge with experimental drug therapies.

Embryonic Stem Cells

Embryonic (ES) stem cells isolated from rodent embryos retain the potential to form embryonic tissues. When ES cells are placed inside a carrier embryo, they resume normal development and contribute to tissues of the live-born animal. ES cells are the preferred cells used in the creation of experimental knockout and knockin rodent strains. Mouse ES cells, such as the mouse 129/SvJ cell line, are derived from the early mouse embryo and are grown under culture conditions well known in the art. Vectors used to produce a transgenic strain contain a disease gene candidate and a marker gen, the latter serves to identify the presence of the introduced disease gene. The vector is transformed into ES cells by methods well known in the art, and transformed ES cells are identified and microinjected into mouse cell blastocysts such as those from the C57BL/6 mouse strain. The blastocysts are surgically transferred to pseudopregnant dams, and the resulting chimeric progeny are genotyped and bred to produce heterozygous or homozygous strains.

ES cells derived from human blastocysts may be manipulated in vitro to differentiate into at least eight separate cell lineages. These lineages are used to study the differentiation of various cell types and tissues in vitro, and they include endoderm, mesoderm, and ectodermal cell types which differentiate into, for example, neural cells, hematopoietic lineages, and cardiomyocytes.

Knockout Analysis

In gene knockout analysis, a region of a mammalian gene is enzymatically modified to include a non-mammalian gene such as the neomycin phosphotransferase gene (neo; Capecchi (1989) Science 244:1288-1292). The modified gene is transformed into cultured ES cells and integrates into the endogenous genome by homologous recombination. The inserted sequence disrupts transcription and translation of the endogenous gene. Transformed cells are injected into rodent blastulae, and the blastulae are implanted into pseudopregnant dams. Transgenic progeny are crossbred to obtain homozygous inbred lines which lack a functional copy of the mammalian gene. In one example, the mammalian gene is a human gene.

Knockin Analysis

ES cells can be used to create knockin humanized animals (pigs) or transgenic animal models (mice or rats) of human diseases. With knockin technology, a region of a human gene is injected into animal ES cells, and the human sequence integrates into the animal cell genome. Transformed cells are injected into blastulae and the blastulae are implanted as described above. Transgenic progeny or inbred lines are studied and treated with potential pharmaceutical agents to obtain information on treatment of the analogous human condition. These methods have been used to model several human diseases.

Non-Human Primate Model

The field of animal testing deals with data and methodology from basic sciences such as physiology, genetics, chemistry, pharmacology and statistics. These data are paramount in evaluating the effects of therapeutic agents on non-human primates as they can be related to human health. Monkeys are used as human surrogates in vaccine and drug evaluations, and their responses are relevant to human exposures under similar conditions. Cynomolgus and Rhesus monkeys (Macaca fascicularis and Macaca mulatta, respectively) and Common Marmosets (Callithrix jacchus) are the most common non-human primates (NHPs) used in these investigations. Since great cost is associated with developing and maintaining a colony of NHPs, early research and toxicological studies are usually carried out in rodent models. In studies using behavioral measures such as drug addiction, NHPs are the first choice test animal. In addition, NHPs and individual humans exhibit differential sensitivities to many drugs and toxins and can be classified as a range of phenotypes from “extensive metabolizers” to “poor metabolizers” of these agents.

In additional embodiments, the polynucleotides which encode the protein may be used in any molecular biology techniques that have yet to be developed, provided the new techniques rely on properties of polynucleotides that are currently known, including, but not limited to, such properties as the triplet genetic code and specific base pair interactions.

### EXAMPLES

### I SEMVNOT04 cDNA Library Construction

The seminal vesicle cDNA library was constructed using tissue isolated from a 61-year-old Caucasian male during a radical prostatectomy. Pathology indicated the seminal vesicles were negative for tumor. Pathology for the associated tumor tissue indicated adenocarcinoma, Gleason grade 3+3, forming a predominant mass involving the right side centrally and peripherally. The tumor invaded the right mid-posterior capsule but did not extend beyond it. The patient presented with induration, hyperplasia of the prostate, and elevated prostate specific antigen. Patient history included renal failure, osteoarthritis, left renal artery stenosis, benign hypertension, thrombocytopenia, hyperlipidemia, and tobacco.

The frozen tissue was homogenized and lysed in TRIZOL reagent (1 gm tissue/10 ml reagent (Invitrogen) using a POLYTRON homogenizer (Brinkmann Instruments, Westbury N.Y.). After a brief incubation on ice, chloroform was added (1:5 v/v), and the lysate was centrifuged. The upper chloroform layer was removed to a fresh tube. and the RNA was extracted with isopropanol, resuspended in DEPC-treated water, and DNAse treated for 25 min at 37° C. The RNA was re-extracted twice with acid phenol-chloroform pH 4.7 and precipitated using 0.3M sodium acetate and 2.5 volumes ethanol. The mRNA was then isolated using the OLIGOTEX kit (Qiagen, Chatsworth Calif.), and cDNA synthesis was initiated using a Notl-oligo d(T) primer. cDNA was blunted, ligated to EcoRI adaptors, digested with NotI, and ligated into the pINCY vector (Incyte Genomics, Palo Alto Calif.).

### II Isolation and Sequencing of cDNA Clones

Plasmid DNA was released from the cells and purified using the REAL Prep 96 plasmid kit (Qiagen). The recommended protocol was employed except for the following changes: 1) the bacteria were cultured in 1 ml of sterile TERRIFIC BROTH (BD Biosciences) with carbenicillin (Carb) at 25 mg/l and glycerol at 0.4%; 2) the cultures were inoculated, incubated for 19 hours, and lysed with 0.3 ml of lysis buffer; and 3) following isopropanol precipitation, the plasmid DNA pellet was resuspended in 0.1 ml of distilled water. After the last step in the protocol, samples were transferred to a 96-well block for storage at 4° C.

The cDNAs were prepared using a MICROLAB 2200 (Hamilton) in combination with DNA ENGINE thermal cyclers (MJ Research) and sequenced by the method of Sanger and Coulson (1975, J Mol Biol 94:441-445) on 377 PRISM DNA Sequencing systems (ABI).

### III Homology Searching of cDNA Clones and Their Deduced Proteins

The nucleotide sequences and/or amino acid sequences of the Sequence Listing were used to query sequences in the GenBank, SwissProt, BLOCKS, and Pima II databases. These databases, which contain previously identified and annotated sequences, were searched for regions of homology using BLAST (Altschul (1993) supra; Altschul (1990) supra).

BLAST produced alignments of both nucleotide and amino acid sequences to determine sequence similarity. Because of the local nature of the alignments, BLAST was especially useful in determining exact matches or in identifying homologs which may be of prokaryotic (bacterial) or eukaryotic (animal, fungal, or plant) origin. Other algorithms could have been used when dealing with primary sequence patterns and secondary structure gap penalties (Smith et al. (1992) Protein Engineering 5:35-51). The sequences disclosed in this application have lengths of at least 49 nucleotides and have no more than 12% uncalled bases (where N is recorded rather than A, C, G, or T).

The BLAST approach searched for matches between a query sequence and a database sequence. BLAST evaluated the statistical significance of any matches found, and reported only those matches that satisfy the user-selected threshold of significance. In this application, threshold was set at 10⁻²⁵ for nucleotides and 10⁻⁸ for peptides.

Incyte nucleotide sequences were searched against the GenBank databases for primate (pri), rodent (rod), and other mammalian sequences (mam), and deduced amino acid sequences from the same clones were then searched against GenBank functional protein databases, mammalian (mamp), vertebrate (vrtp), and eukaryote (eukp), for homology.

### IV Northern Analysis

Northern analysis is a laboratory technique used to detect the presence of a transcript of a gene and involves the hybridization of a labeled nucleotide sequence to a membrane on which RNAs from a particular cell type or tissue have been bound. (See, e.g., Sambrook, supra, ch. 7; Ausubel supra).

Transcript Image

A transcript image was produced using the LIFESEQ GOLD database (Jan02rel, Incyte Genomics). TI allowed assessment of the relative abundance of SEQ ID NO:2 in all cDNA libraries of the database. Although criteria for transcript imaging can be selected from category, number of cDNAs per library, library description, disease indication, clinical relevance of sample, and the like, no limitations were place on the data below. Zweiger (2001) Transducing the Genome. McGraw Hill, San Francisco Calif.) and Glavas et al. (2001, Proc Natl Acad Sci 6319-6324) discuss the correspondence between mRNA and protein expression.

In the LIFESEQ databases, all cDNAs and their libraries have been categorized by system, organ/tissue and cell type. For each category, the number of libraries in which the sequence was expressed was counted and shown over the total number of libraries in that category. For each library, the number of cDNAs were also counted and shown over the total number of cDNAs in that library. In some transcript images, all normalized or subtracted libraries, which have high copy number sequences removed prior to processing, and all mixed or pooled tissues, which are considered non-specific in that they contain more than one tissue type or more than one subject's tissue, can be excluded from the analysis. Treated and untreated cell lines and/or fetal tissue data can also be excluded where clinical relevance is emphasized. Conversely, fetal tissue can be emphasized wherever elucidation of inherited disorders or differentiation of particular adult or embryonic stem cells into tissues or organs such as heart, kidney, nerves or pancreas would be aided by removing clinical samples from the analysis. Transcript imaging is used to support data from other methodologies such as guilt-by-association and various PCR and hybridization analyses including arrays.

The transcript image for SEQ ID NO:2 in prostate is shown below. The first column shows library name; the second column, the number of cDNAs sequenced in that library; the third column, the description of the library: the fourth column, absolute abundance of the transcript in the library; and the fifth column, percentage abundance of the transcript in the library.

SEQ ID NO:2

Category: Male Reproductive (Prostate)

| | | Description of | Abun- | |
|---|---|---|---|---|
| Library | cDNAs | Tissue | dance | % Abundance |
| PROSTUT18 | 2201 | adenoCA, 68M, | 1 | 0.0454 |
| | | m/PROSTMT03 | | |
| PROSTUS20 | 4546 | adenoCA, 59M, | 2 | 0.0440 |
| | | SUB, | | |
| | | m/PROSNOT19 | | |
| PROSTUT10 | 6969 | adenoCA, 66M, | 3 | 0.0430 |
| | | m/PROSNOT15, | | |
| | | PROSDIN01 | | |

SEQ ID NO:2 was differentially expressed in the adenocarcinoma of the prostate and not expressed in matched (m/) cytologically normal tissue. It was not as significantly expressed in libraries (PROSNOT07, PROSTMT07, and PROSTMY01) from prostate with adenofibromatous hyperplasia (AH), a common precursor to adenocarcinoma or in two matched (m/) cytologically normal libraries (PROSTUT05, PROSTUT16). SEQ ID NO:2 was not significantly expressed in four libraries (PROSBPS05, PROSDIP02, PROSDIP03, and PROSBPT03) made from tissue displaying benign prostatic hyperplasia (BPH) or in five libraries (PROETMP02, PROETMP03, PROETMP04, PROETMP06, and PROETMP07) made from tissue displaying prostatic intraepithelial neoplasia (PIN). When used in a tissue specific and clinically relevant manner, SEQ ID NO:2 or an antibody specifically binding the encoded protein may be used with biopsied tissue to diagnose cancer of the prostate, specifically prostatic adenocarcinoma.

Category: Hemic/Immune (spleen)

| | | Description of | | % |
|---|---|---|---|---|
| Library | cDNAs | Tissue | Abundance | Abundance |
| SPLNTUT02 | 3077 | spleen tumor, | 2 | 0.0650 |
| | | Hodgkin's, 45M | | |
| SPLNFET01 | 2731 | spleen, fetal, | 1 | 0.0366 |
| | | pool | | |
| SPLNNOT12 | 3869 | spleen, | 1 | 0.0258 |
| | | aw/pancreas | | |
| | | neuroendocrine | | |
| | | CA, 65F | | |
| SPLNFET02 | 7854 | spleen, fetal, | 2 | 0.0255 |
| | | 23 wM | | |
| SPLNNOT04 | 10172 | spleen, 2M | 2 | 0.0197 |

SEQ ID NO:2 was differentially expressed in spleen of a patient diagnosed with Hodgkin's disease when compared with all other splenic tissues; it was not expressed in cytologically normal adult spleen libraries, SPLNNOP01, SPLNNOP03 and SPLNNOT02.

The description of the SPLNTUT02 cDNA library is as follows: The cDNA library was constructed using polyA RNA isolated from spleen tumor tissue removed from a 45-year-old male during a staging laparotomy. Pathology indicated nodular sclerosing type of Hodgkin's disease forming innumerable nodules. Multiple lymph nodes were positive for Hodgkin's disease. Liver biopsies were negative, and the patient was not taking any medication.

When used in a tissue specific and clinically relevant manner, SEQ ID NO:2 or an antibody specifically binding the encoded protein may be used with biopsied tissue to diagnose Hodgkin's disease.

### V Extension of PINCH-PH Encoding Polynucleotides

The nucleic acid sequence of Incyte Clone 3540806 was used to design oligonucleotide primers for extending a partial nucleotide sequence to full length. One primer was synthesized to initiate extension of an antisense polynucleotide, and the other was synthesized to initiate extension of a sense polynucleotide. Primers were used to facilitate the extension of the known sequence “outward” generating amplicons containing new unknown nucleotide sequence for the region of interest. The initial primers were designed from the cDNA using OLIGO software (Molecular Insights), or another program, to be about 22 to 30 nucleotides in length, to have a GC content of about 50% or more, and to anneal to the target sequence at temperatures of about 68° C. to about 72° C. Any stretch of nucleotides which would result in hairpin structures and primer-primer dimerizations was avoided.

Selected human cDNA libraries were used to extend the sequence. If more than one extension is necessary or desired, additional sets of primers are designed to further extend the known region.

High fidelity amplification was obtained by following the instructions for the XL-PCR kit (ABI) and thoroughly mixing the enzyme and reaction mix. PCR was performed using the DNA ENGINE thermal cycler (MJ Research) beginning with 40 pmol of each primer and the recommended concentrations of all other components of the kit, with the following parameters: Step 1, 94° C. for 1 min (initial denaturation); Step 2, 65° C. for 1 min; Step 3, 68° C. for 6 min; Step 4, 94° C. for 15 sec; Step 5, 65° C. for 1 min; Step 6, 68° C. for 7 min; Step 7, repeat steps 4 through 6 for an additional 15 cycles; Step 8, 94° C. for 15 sec; Step 9, 65° C. for 1 min; Step 10, 68° C. for 7:15 min; Step 11, repeat steps 8 through 10 for an additional 12 cycles; Step 12, 72° C. for 8 min; and Step 13, 4° C. (and holding).

A 5 μl to 10 μl aliquot of the reaction mixture was analyzed by electrophoresis on a low concentration (about 0.6% to 0.8%) agarose mini-gel to determine which reactions were successful in extending the sequence. Bands thought to contain the largest products were excised from the gel, purified using QIAQuick resin (Qiagen), and trimmed of overhangs using Klenow enzyme to facilitate religation and cloning.

After ethanol precipitation, the products were redissolved in 13 μl of ligation buffer, 1 μl T4-DNA ligase (15 units) and 1 μl T4 polynucleotide kinase were added, and the mixture was incubated at room temperature for 2 to 3 hours, or overnight at 16° C. Competent E. coli cells (suspended in 40 μl of media) were transformed with 3 μl of ligation mixture and cultured in 80 μl of SOC medium (Sambrook, supra, Appendix A, p. 2). After incubation for one hour at 37° C., the E. coli mixture was plated on Luria Bertani (LB) agar (Ausubel, supra, p. 1-3) containing 2× Carb. The following day, several colonies were randomly picked from each plate and cultured in 150 μl of liquid LB/2× Carb medium placed in an individual well of a commercially-available sterile 96-well microtiter plate. The following day, 5 μl of each overnight culture was transferred into a non-sterile 96-well plate and, after dilution 1:10 with water, 5 μl from each sample was transferred into a PCR array.

For PCR amplification, 18 μl of concentrated PCR reaction mix (3.3×) containing 4 units of rTth DNA polymerase, a vector primer, and one or both of the gene specific primers used for the extension reaction were added to each well. Amplification was performed using the following conditions: Step 1, 94° C. for 60 sec; Step 2, 94° C. for 20 sec; Step 3, 55° C. for 30 sec; Step 4, 72° C. for 90 sec; Step 5, repeat steps 2 through 4 for an additional 29 cycles; Step 6, 72° C. for 180 sec; and Step 7, 4° C. (and holding).

Aliquots of the PCR reactions were run on agarose gels together with molecular weight markers. The sizes of the PCR products were compared to the original partial cDNAs; clones were selected, ligated into plasmid, and sequenced.

In like manner, the nucleotide sequence of SEQ ID NO:2 is used to obtain 5′ regulatory sequences using the procedure above, oligonucleotides designed for 5′ extension, and a genomic library.

### VI Labeling and Use of Individual Hybridization Probes

Hybridization probes derived from SEQ ID NO:2 are employed to screen cDNAs, genomic DNAs, or mRNAs. Although the labeling of oligonucleotides, consisting of about 20 base pairs, is specifically described, the same procedure is used with larger nucleotide fragments. Oligonucleotides are designed using state-of-the-art software such as OLIGO software (Molecular Insights) and labeled by combining 50 pmol of each oligomer, 250 μCi of [γ-³²P] adenosine triphosphate (APB), and T4 polynucleotide kinase (NEN Life Science Products, Boston Mass.). The labeled oligonucleotides are purified using a SEPHADEX G-25 superfine resin column (APB). An aliquot containing 10⁷ counts per minute of the labeled probe is used in a typical membrane-based hybridization analysis of human genomic DNA digested with one of the following endonucleases: Ase I, Bgl II, Eco RI, Pst I, Xba 1, or Pvu II (NEN Life Science Products).

The DNA from each digest is fractionated on a 0.7 percent agarose gel and transferred to NYTRAN PLUS membranes (Schleicher & Schuell, Durham N.H.). Hybridization is carried out for 16 hours at 40° C. To remove nonspecific signals, blots are sequentially washed at room temperature under increasingly stringent conditions up to 0.1× saline sodium citrate and 0.5% sodium dodecyl sulfate. After XOMAT-AR film (Eastman Kodak, Rochester N.Y.) is exposed to the blots for several hours, hybridization patterns are compared visually.

### VII Microarrays

To produce oligonucleotides for a microarray, one of the nucleotide sequences of the present invention is examined using a computer algorithm which starts at the 3′ end of the nucleotide sequence. For each, the algorithm identifies oligomers of defined length that are unique to the nucleic acid sequence, have a GC content within a range for hybridization, and lack secondary structure that would interfere with hybridization. The algorithm identifies approximately 20 oligonucleotides corresponding to each nucleic acid sequence. For each sequence-specific oligonucleotide, a pair of oligonucleotides is synthesized in which the first oligonucleotides differs from the second oligonucleotide by one nucleotide in the center of the sequence. The oligonucleotide pairs can be arranged on a substrate, e.g. a silicon chip, using a light-directed chemical process (Chee, supra).

In the alternative, a chemical coupling procedure and an ink jet device can be used to synthesize oligomers on the surface of a substrate (Baldeschweiler, supra). An array analogous to a dot or slot blot may also be used to arrange and link fragments or oligonucleotides to the surface of a substrate using or thermal, UV, mechanical, or chemical bonding procedures, or a vacuum system. A typical array may be produced by hand or using commercially available methods and machines and contain any number of elements. After hybridization, nonhybridized probes are removed and a scanner used to determine the levels and patterns of fluorescence. The degree of complementarity and the relative abundance of each oligonucleotide sequence on the microarray may be assessed through analysis of the scanned images.

### VIII Complementary Polynucleotides

Sequences complementary to the PINCH-PH-encoding sequences, or any parts thereof, are used to detect, decrease, or inhibit expression of naturally occurring PINCH-PH. Although use of oligonucleotides comprising from about 15 to 30 base pairs is described, the same procedure is used with smaller or with larger sequence fragments. Oligonucleotides are designed using OLIGO software (Molecular Insights) and the coding sequence of PINCH-PH. To inhibit transcription, a complementary oligonucleotide is designed from the most unique 5′ sequence and used to prevent promoter binding to the coding sequence. To inhibit translation, a complementary oligonucleotide is designed to prevent ribosomal binding to the PINCH-PH-encoding transcript.

### IX Expression of PINCH-PH

Expression of PINCH-PH is accomplished by subcloning the cDNA into a vector and transforming the vector into host cells. The vector contains a promoter upstream of the cloning site operably associated with the cDNA of interest (Sambrook, supra, pp. 404-433; Rosenberg et al. (1983) Methods Enzymol 101:123-138).

Expression and purification of the protein are achieved using either a mammalian cell expression system or an insect cell expression system. The pUB6/V5-His vector system (Invitrogen) is used to express the protein in CHO cells. The vector contains the selectable bsd gene, multiple cloning sites, the promoter/enhancer sequence from the human ubiquitin C gene, a C-terminal V5 epitope for antibody detection with anti-V5 antibodies, and a C-terminal polyhistidine (6×His) sequence for rapid purification on PROBOND resin (Invitrogen). Transformed cells are selected on media containing blasticidin.

Spodoptera frugiperda (Sf9) insect cells are infected with recombinant Autographica californica nuclear polyhedrosis virus (baculovirus). The polyhedrin gene is replaced with the polynucleotide by homologous recombination and the polyhedrin promoter drives transcription. The protein is synthesized as a fusion protein with 6×his which enables purification as described above. Purified protein is used in the following activity and to make antibodies.

### X Demonstration of PINCH-PH Activity

The activity of PINCH-PH is determined by its ability to promote differentiation of permeabilized C2 muscle cells. The basis of this assay lies in the ability of LIM-only proteins to substitute for muscle LIM protein (MLP) in promoting the differentiation of mouse C2 myogenic cells. Shifting C2 cells from high serum medium to low-serum medium induces differentiation of these cells, wherein they change from round cells to spindle-shaped cells. In addition, the cells express myotubules and other cytoskeletal components characteristic of a mature muscle cell. C2 cells which have been stably transfected with a vector expressing antisense to the MLP message (C2-AS cells) do not undergo differentiation following a shift to low-serum media. However, these cells can be induced to undergo differentiation under these conditions provided they are permeabilized and exposed to purified MLP or transiently transfected with a vector expressing MLP. In addition, other LIM-only proteins including Drosophila homolog of MLP (DMLP) and cysteine-rich intestinal protein (CRIP), are able to substitute for MLP in promoting differentiation of C2-AS cells. Thus, the activity of a sample containing PINCH-PH is assayed by determining its ability to promote differentiation in C2-AS cells. Following permeabilization and treatment with PINCH-PH-containing samples, the degree of differentiation of C2-AS cells is measured by visual examination, e.g., scoring the cells for the change in morphology characteristic of differentiated C2-AS cells (Arber et al. (1994) Cell 79:221-231).

### XI Production of Antibodies Which Specifically Bind the Protein

Purification using polyacrylamide gel electrophoresis or similar techniques is used to isolate protein for immunization of hosts or host cells to produce antibodies which specifically bind PINCH-PH using standard protocols.

Alternatively, the amino acid sequence of the protein is analyzed using readily available commercial software to determine regions of high immunogenicity. A peptide with high immunogenicity is cleaved, recombinantly-produced, or synthesized and used to raise antibodies by means known to those of skill in the art. Methods for selection of appropriate antigenic determinants such as those near the C-terminus or in hydrophilic regions are well described in the art (Ausubel (1997) supra, Chap. 11). An immunogenic region extends from C₁₅ through F₅₀ or from C₇₆ through L₁₀₉ of SEQ ID NO:1.

Oligopeptides of about 15 residues in length are synthesized using an 431 A peptide synthesizer (ABI) using FMOC chemistry and coupled to carriers such as BSA, thyroglobulin, or KLH (Sigma-Aldrich) by reaction with N-maleimidobenzoyl-N-hydroxysuccinimide ester to increase immunogenicity. The coupled peptide is then used to immunize the host. Rabbits are immunized with the oligopeptide-KLH complex in complete Freund's adjuvant. Resulting antisera are tested for antipeptide activity by binding the peptide to a substrate, blocking with 1% BSA, reacting with rabbit antisera, washing, and reacting with radio-iodinated goat anti-rabbit IgG.

### XII Immunopurification Using Antibodies

Naturally occurring or recombinantly produced protein is purified by immunoaffinity chromatography using antibodies which specifically bind the protein. An immunoaffinity column is constructed by covalently coupling the antibody to CNBr-activated SEPHAROSE resin (APB). Media containing the protein is passed over the immunoaffinity column, and the column is washed using high ionic strength buffers in the presence of detergent to allow preferential absorbance of the protein. After coupling, the protein is eluted from the column using a buffer of pH 2-3 or a high concentration of urea or thiocyanate ion to disrupt antibody/protein binding, and the purified protein is collected.

### XIII Antibody Arrays

Protein:protein Interactions

In an alternative to yeast two hybrid system analysis of proteins, an antibody array can be used to study protein-protein interactions and phosphorylation. A variety of protein ligands are immobilized on a membrane using methods well known in the art. The array is incubated in the presence of cell lysate until protein:antibody complexes are formed. Proteins of interest are identified by exposing the membrane to an antibody specific to the protein of interest. In the alternative, a protein of interest is labeled with digoxigenin (DIG) and exposed to the membrane; then the membrane is exposed to anti-DIG antibody which reveals where the protein of interest forms a complex. The identity of the proteins with which the protein of interest interacts is determined by the position of the protein of interest on the membrane.

Proteomic Profiles

Antibody arrays can also be used for high-throughput screening of recombinant antibodies. Bacteria containing antibody genes are robotically-picked and gridded at high density (up to 18,342 different double-spotted clones) on a filter. Up to 15 antigens at a time are used to screen for clones to identify those that express binding antibody fragments. These antibody arrays can also be used to identify proteins which are differentially expressed in samples (de Wildt, supra).

### XIV Identification of Molecules Which Interact with PINCH-PH

PINCH-PH, or biologically active fragments thereof, are labeled with ¹²⁵I Bolton-Hunter reagent (Bolton et al. (1973) Biochem J 133:529-539). Candidate molecules previously arrayed in the wells of a multi-well plate are incubated with the labeled PINCH-PH, washed, and any wells with labeled PINCH-PH complex are assayed. Data obtained using different concentrations of PINCH-PH are used to calculate values for the number, affinity, and association of PINCH-PH with the candidate molecules.

Various modifications and variations of the described methods and systems of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

What is claimed is:

1. A substantially purified human Particularly Interesting New Cyc-His protein homolog (PINCH-PH) comprising a protein having an amino acid sequence of SEQ ID NO:1.

2. A purified antibody which specifically binds to the protein of claim 1.

3. The antibody of claim 2, wherein the antibody is selected from a polyclonal antibody, a monoclonal antibody, a chimeric antibody, a recombinant antibody, a humanized antibody, a single chain antibody, a Fab fragment, an F(ab′)₂ fragment, an Fv fragment; and an antibody-peptide fusion protein.

4. A method of making a polyclonal antibody which specifically binds a protein, the method comprising:
a) immunizing a animal with a protein having the amino acid sequence of SEQ ID NO:1 under conditions to elicit an antibody response;
b) isolating animal antibodies;
c) attaching the protein to a substrate;
d) contacting the substrate with isolated antibodies under conditions to form an antibody:protein complex;
e) dissociating the antibodies from the complex so formed, thereby obtaining polyclonal antibodies with the specificity of the antibody of claim 2.

5. A polyclonal antibody produced by the method of claim 4.

6. A method of preparing a monoclonal antibody which specifically binds a protein, the method comprising:
a) immunizing a animal with a protein having the amino acid sequence of SEQ ID NO:1 under conditions to elicit an antibody response;
b) isolating antibody-producing cells from the animal;
c) fusing the antibody-producing cells with immortalized cells in culture to form monoclonal antibody producing hybridoma cells;
d) culturing the hybridoma cells; and
e) isolating monoclonal antibodies from culture.

7. A monoclonal antibody produced by the method of claim 6.

8. A method for using an antibody to immunopurify a protein comprising:
a) attaching the antibody of claim 2 to a substrate,
b) exposing the antibody to a sample containing protein under conditions to allow antibody:protein complexes to form,
c) dissociating the protein from the complex, and
d) collecting the purified protein.

9. A method for using an antibody to detect expression of a protein in a sample, the method comprising:
a) combining the antibody of claim 2 with a sample under conditions which allow the formation of antibody:protein complexes; and
b) detecting complex formation, wherein complex formation indicates expression of the protein in the sample.

10. The method of claim 9 wherein the sample is biopsied tissue.

11. The method of claim 9 wherein the complex formation is compared with standards and is diagnostic of prostatic adenocarcinoma.

12. The method of claim 9 wherein the complex formation is compared with standards and is diagnostic of Hodgkin's disease.

13. A composition comprising an antibody of claim 2 and a labeling moiety.

14. A composition comprising an antibody of claim 2 and a pharmaceutical agent.

15. A method for treating prostatic adenocarcinoma, the method comprising administering the antibody of claim 2 to a subject in need of such treatment.

16. A method for treating a cancer, the method comprising administering the antibody of claim 2 to a subject in need of such treatment.

17. A purified agonist which specifically binds the protein of claim 1.

18. A purified antagonist which specifically binds the protein of claim 1.


SEQUENCE LISTING (3 sequences)

SEQ ID NO: 1 - protein, 341 aa, Homo sapiens
  misc_feature: Incyte ID No: 3540806CD1
MTGSNMSDALANAVCQRCQARFSPAERIVNSNGELYHEHCFVCAQCFRPFPEGLFYEFEGRKYCEHDFQMLFAPCCGSCGEFIIGRVIKAMNNNWHPGCFRCELCDVELADLGFVKNAGRHLCRPCHNREKAKGLGKYICQRCHLVIDEQPLMFRSDAYHPDHFNCTHCGKELTAEARELKGELYCLPCHDKMGVPICGACRRPIEGRVVNALGKQWHVEHFVCAKCEKPFLGHRHYEKKGLAYCETHYNQLFGDVCYNCSHVIEGDVVSALNKAWCVSCFSCSTCNSKLTLKNKFVEFDMKPVCKRCYEKFPLELKKRLKKLSELTSRKAQPKATDLNSA

SEQ ID NO: 2 - DNA, 1959 bp, Homo sapiens
  misc_feature: Incyte ID No: 3540806CB1
tgcagcagcagagggagacccgcggcaaccccggcaacccagggctcggcgtcgctgccaccatgacgggaagcaatatgtcggacgccttggccaacgccgtgtgccagcgctgccaggcccgcttctcccccgccgagcgcattgtcaacagcaatggggagctgtaccatgagcactgcttcgtgtgtgcccagtgcttccggcccttccccgaggggctcttctatgagtttgaaggccggaagtactgcgaacacgacttccaaatgctgtttgctccgtgctgtggatcctgcggtgagttcatcattggccgcgtcatcaaggccatgaacaacaactggcacccgggctgcttccgctgcgagctgtgtgatgtggagctggctgacctgggctttgtgaagaatgccggcaggcatctctgccggccttgccacaaccgtgagaaggccaagggcctgggcaagtacatctgccagcggtgccacctggtcatcgacgagcagcccctcatgttcaggagcgacgcctaccaccctgaccacttcaactgcacccactgtgggaaggagctgacagccgaggcccgcgagctgaagggtgagctctactgcctgccctgccatgacaagatgggcgtccccatctgcggggcctgccgccggcccatcgagggccgagtggtcaacgcgctgggcaagcagtggcacgtggagcactttgtctgtgccaagtgtgagaagccattcctggggcaccggcactatgagaagaagggcctggcctactgcgagactcactacaaccagctcttcggggacgtctgctacaactgcagccatgtgattgaaggcgatgtggtgtcggccctcaacaaggcctggtgtgtgagctgcttctcctgctccacctgcaacagcaagctcaccctgaagaacaagtttgtggagttcgacatgaagcccgtgtgtaagaggtgctacgagaagttcccgctggagctgaagaagcggctgaagaagctgtcggagctgacctcccgcaaggcccagcccaaggccacagacctcaactctgcctgaaggccctcttgcgcactgcctctcggcccctccgccttctcccctcctgctgtccatgcttggccccctcgtccccatccacctgtgccctccgcatcttaccctccctttctctttcctcattgccttctcccttcctgttccctcatctctgccttccccatgtctctcctctccttggccgtggcttctgtctgtgaggaggcaggagctggggagtgggagcctatgaccccacgtctgacagccatgtccacctgtgcccacagcttccgcccacagacctccagggacaggagcaaattgcaccacagctccccgcctggcctggccctccccaggcggctcagtggctcatgctgtcctgtgagagcccctgccccagagcggccccactaagcgcatgtggctcctgggctacccacagccagggcagcctgctggagccacagggccagggccatgcagatggaggcctctgggagccacctccaatccctcaccactcactcaaccagtggcacagtgtccttgtgcccacactgagccagcaagtcctgctgtccacacccacaagctacctggagggacaggacccacctccatccttcggaaggccttcctggaatcccaccttggcctccgccctcggttccgccccgcccctctccccccgaccttggggcttgtgtcgagcccttgggtggggccaggaggaggtgatggcgtcagaggaggtgtggtcagaggtgacttgttcccacctccagggaggacgcttcgtcttcggccagcgcagacctggtgtttgtttgtttgttgggtcacgcttgcacaatgaaggcttgttcacacaaaaaaaaaaaaaaaa

SEQ ID NO: 3 - protein, 314 aa, Homo sapiens
  ref <308>: DATABASE ACCESSION NUMBER: Genbank ID No: g516012
  ref <309>: DATABASE ENTRY DATE: 1994-07-26
MANALASATCERCKGGFAPAEKIVNSNGELYHEQCFVCAQCFQQFPEGLFYEFEGRKYCEHDFQMLFAPCCHQCGEFIIGRVIKAMNNSWHPECFRCDLCQEVLADIGFVKNAGRHLCRPCHNREKARGLGKYICQKCHAIIDEQPLIFKNDPYHPDHFNCANCGKELTADARELKGELYCLPCHDKMGVPICGACRRPIEGRVVNAMGKQWHVEHFVCAKCEKPFLGHRHYERKGLAYCETHYNQLFGDVCFHCNRVIEGDVVSALNKAWCVNCFACSTCNTKLTLKNKFVEFDMKPVCKKCYEISIGAEEKT